(19) United States
(12) Patent Application Publication
(10) Pub. No.: US 2009/0325248 A1
(43) Pub. Date: Dec. 31, 2009

(54) Microbiological Production of 3-Hydroxypropionic Acid

(75) Inventors: Achim Marx, Gelnhausen (DE); Volker F. Wendisch, Juelich (DE); Doris Rittmann, Juelich (DE); Stefan Buchholz, Hanau (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Evonik Degussa GmbH, Essen (DE)

(21) Appl. No.: 12/067,266

(22) PCT Filed: Oct. 9, 2006
(86) PCT No.: PCT/EP2006/067182

§ 371 Date: Mar. 18, 2008

(30) Foreign Application Priority Data
Oct. 10, 2005 (DE) 102005048818.8

(51) Int. Cl. C12P 7/52; C12N 1/20; C12N 15/74

(52) U.S. Cl. 435/141; 435/252.3; 435/471

(57) ABSTRACT

The present invention relates to a cell which is genetically modified in relation to its wild type and which exhibits at least one of the properties a) or b):
a) an increased activity by comparison with its wild type of an enzyme E₁ which catalyzes the conversion of pyruvate into oxaloacetate, or of an enzyme E_{1b} which catalyzes the conversion of phosphoenolpyruvate into oxaloacetate,
b) an increased activity by comparison with its wild type of an enzyme E₂ which catalyzes the conversion of aspartate into beta-alanine,
   where, besides properties a) or b), the cell is characterized by at least one of the properties c) or d)
c) the genetically modified cell is able to export beta-alanine out of the cell,
d) the genetically modified cell is able to convert beta-alanine into 3-hydroxypropionic acid.

The invention also relates to methods for producing a genetically modified cell, to the genetically modified cells obtainable by this method, to methods for producing 3-hydroxypropionic acid, to a method for producing acrylic acid, to a method for producing polyacrylates, to a method for producing acrylic esters, and to the use of cells for producing 3-hydroxypropionic acid.

The present invention relates to a cell which is genetically modified in relation to its wild type, to a method for producing a genetically modified cell, to the genetically modified cells obtainable by this method, to methods for producing 3-hydroxypropionic acid, to a method for producing polyacrylates, to a method for producing acrylic esters, and to the use of cells for producing 3-hydroxypropionic acid.

Acrylic acid is a starting compound which is very important industrially. It is used inter alia for producing polyacrylates, especially crosslinked, partially neutralized polyacrylates which, in the dry and in the substantially anhydrous state, exhibit a great ability to absorb water. These crosslinked polyacrylates, which are referred to as “superabsorbents”, are able to absorb a multiple of their own weight of water. Because of the great absorbency, the absorbing polymers are suitable for incorporation into water-absorbing structures and articles such as, for example, diapers, incontinence products or sanitary napkins. In this connection, reference is made to Modern Superabsorbent Polymer Technology; F. L. Buchholz, A. T. Graham, Wiley-VCH, 1998 .

Acrylic esters such as, for example, methyl acrylate and butyl acrylate are likewise starting compounds of industrial importance which are employed in particular for producing copolymers. These copolymers are usually employed in the form of polymer dispersions as adhesives, paints or textile, leather and paper auxiliaries.

Acrylic acid is produced industrially primarily by the two-stage, catalytic gas-phase oxidation of propylene, the propylene in turn being obtained by thermal cleavage of benzines resulting from petroleum processing. The acrylic acid obtained in this way is subsequently esterified where appropriate by adding alcohols.

Disadvantages of the two-stage method for producing acrylic acid are firstly that the temperatures of between 300 and 450° C. used in both stages lead to the formation of oligomers and further unwanted cracking products. This results in an undesirably large amount of higher-boiling compounds than acrylic acid, or of compounds which can be separated from acrylic acid only with difficulty, such as, for instance, acetic acid. These compounds must usually be removed by distillation from the acrylic acid, in turn leading to a further thermal stress on the acrylic acid and the formation, associated therewith, of dimers and oligomers. A high content of acrylic acid dimers or acrylic acid oligomers is, however, disadvantageous because when superabsorbents are produced by free-radical polymerization of acrylic acid in the presence of crosslinkers, these dimers or oligomers are incorporated into the polymer. However, during the post-treatment of the surface of the polymer particles, which takes place following the polymerization, for example during a surface post-crosslinking, the dimers incorporated into the polymer are cleaved to form β-hydroxypropionic acid, which is dehydrated under the post-crosslinking conditions to form acrylic acid. A high content of dimeric acrylic acid in the acrylic acid employed to produce the superabsorbents therefore leads to the content of acrylic acid monomers increasing during a thermal treatment of the polymers, like that taking place during the post-crosslinking.

Other, often toxic, compounds are also detectable in the acrylic acid obtainable by catalytic gas-phase oxidation. These impurities include in particular aldehydes which interfere with the progress of the polymerization, resulting in the polymers still containing considerable amounts of soluble constituents.

Some approaches to solving these problems have already been described in the prior art (see, for example, EP-A 0 574 260 or DE-A 101 38 150).

A further disadvantage of this conventional method for producing acrylic acid is that the precursor employed (propylene) is produced from petroleum and thus from non-renewable raw materials, this being a matter for concern from the economic viewpoint, especially in the long term, especially in view of the increasing difficulty and especially increasing costs of extracting petroleum.

In this connection, some approaches have also been described in the prior art for countering this problem.

Thus, WO-A 03/62173 describes the production of acrylic acid with initial fermentative formation from pyruvate of alpha-alanine which is then converted into beta-alanine by the enzyme 2,3-aminomutase. The beta-alanine in turn is converted via β-alanyl-CoA, acrylyl-COA, 3-hydroxypropionyl-CoA or else via malonic semialdehyde into 3-hydroxypropionic acid, from which acrylic acid is obtained following a dehydration.

WO-A 02/42418 describes a further route for producing, for example, 3-hydroxypropionic acid from renewable raw materials. In this case, pyruvate is initially converted into lactate, from which lactyl-CoA is subsequently formed. The lactyl-CoA is then converted via acrylyl-CoA and 3-hydroxypropionyl-CoA into 3-hydroxypropionic acid. A further route described in WO-A 02/42418 for producing 3-hydroxypropionic acid envisages the conversion of glucose via propionate, propionyl-COA, acrylyl-CoA and 3-hydroxypropionyl-CoA. This publication also describes the conversion of pyruvate into 3-hydroxypropionic acid via acetyl-CoA and malonyl-CoA. The 3-hydroxypropionic acid obtained via the respective routes can be converted into acrylic acid by dehydration.

WO-A 01/16346 describes the fermentative production of 3-hydroxypropionic acid from glycerol, employing micro-organisms which express the dhaB gene from Klebsiella pneumoniae (a gene which codes for glycerol dehydratase) and a gene which codes for an aldehyde dehydrogenase. In this way there is formation from glycerol, via 3-hydroxypropionaldehyde, of 3-hydroxy-propionic acid which can then be converted by dehydration into acrylic acid.

The disadvantage of the fermentative method described above for producing 3-hydroxypropionic acid as starting compounds for the synthesis of acrylic acid is inter alia that the amount of 3-hydroxypropionic acid formed in the fermentation solution is too small for this fermentation solution to be used as starting material for the industrial production of acrylic acid in an economically advantageous manner.

The present invention was based on the object of overcoming the disadvantages emerging from the prior art.

The present invention was based on the object in particular of providing recombinant microorganisms or systems composed of at least two recombinant microorganisms which are able even better, especially even more efficiently than the microorganisms described in the prior art, to produce from renewable raw materials, especially from carbohydrates and/or from glycerol, 3-hydroxypropionic acid which can then be converted in a mild dehydration reaction into pure acrylic acid.

A contribution to achieving the aforementioned objects is provided by a cell which is genetically modified in relation to its wild type and which exhibits at least one, preferably both, of the properties a) and b):
a) an activity, which is increased by comparison with its wild type, preferably by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of an enzyme E₁ₐ which catalyzes the conversion of pyruvate into oxaloacetate, or of an enzyme E_{1b} which catalyzes the conversion of phosphoenolpyruvate into oxaloacetate, but preferably of an enzyme E₁ₐ which catalyzes the conversion of pyruvate into oxalo-acetate,
b) an activity, which is increased by comparison with its wild type, preferably by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of an enzyme E₂ which catalyzes the conversion of aspartate into β-alanine,
   where, besides properties a) or b), preferably a) and b), the cell is characterized by at least one of the properties c) or d):
c) the genetically modified cell is able to export β-alanine out of the cell;
d) the genetically modified cell is able to convert β-alanine into 3-hydroxypropionic acid.

A cell genetically modified in this way is able, itself or in combination with other cells which can convert β-alanine into 3-hydroxypropionic acid, to form 3-hydroxypropionic acid from carbohydrates or from glycerol, because the β-alanine formed can be converted into 3-hydroxypropionic acid.

A “wild type” of a cell preferably refers to a cell whose genome is in a condition such as results naturally through evolution. The term is used both for the whole cell and for individual genes. The term “wild type” therefore does not encompass in particular those cells or those genes whose gene sequences have, at least in part, undergone modification by a man, using recombinant methods.

The term “increased activity of an enzyme” preferably means an increased intracellular activity. The wording “an activity which is increased in relation to its wild type of an enzyme” also encompasses in particular a cell whose wild type exhibits no, or at least no detectable, activity of this enzyme and which shows a detectable activity of this enzyme only after increasing the enzymatic activity, for example by over-expression. In this connection, the term “overexpression” or the wording used in the following statement “increasing expression” also encompasses the case where an initial cell, for example a wild-type cell, exhibits no, or at least no detectable, expression and a detectable expression of the enzyme is induced only by recombinant methods.

It is possible in principle to achieve an increase in the enzymatic activity by increasing the copy number of the gene sequence or gene sequences which code for the enzyme, by using a strong promoter or by utilizing a gene or allele which codes for a corresponding enzyme having an increased activity and, where appropriate, by combining these measures. Cells genetically modified according to the invention are generated for example by transformation, transduction, conjugation or a combination of these methods with a vector which comprises the desired gene, an allele of this gene or parts thereof and a vector which enables expression of the gene. Heterologous expression is achieved in particular by integrating the gene or the alleles into the chromosome of the cell or an extrachromosomally replicating vector.

A survey of the possible ways of increasing the enzymatic activity in cells is given, for the example of pyruvate carboxylase, in DE-A-100 31 999, which is introduced hereby as reference and whose disclosure in relation to the possible ways of increasing enzymatic activity in cells forms part of the disclosure of the present invention.

Expression of the enzymes and genes mentioned above and all those mentioned hereinafter can be detected with the aid of one- and two-dimensional protein gel fractionation and subsequent optical identification of the protein concentration with appropriate analysis software in the gel. If the increase in an enzymatic activity is based exclusively on an increase in the expression of the corresponding gene, the quantification of the increase in the enzymatic activity can be determined in a simple manner by comparing the one- or two-dimensional protein fractionations between wild type and genetically modified cell. A useful method for preparing the protein gels in the case of coryneform bacteria and for identifying the proteins is the procedure described by Hermann et al. ((2001) Electrophoresis 22: 1712-1723). The protein concentration can likewise be analyzed by Western blot hybridization with an antibody which is specific for the protein to be detected (Sambrook et al., Molecular Cloning: a laboratory manual, 2^{nd} Ed. Cold Spring Harbor Laboratory Press, Cold Spring Harbor, N.Y. USA, 1989) and subsequent optical analysis with appropriate software for concentration determination (Lohaus und Meyer, (1989) Biospektrum 5: 32-39; Lottspeich (1999) Angewandte Chemie 111: 2630-2647). The activity of DNA-binding proteins can be measured by means of DNA band-shift assays (also referred to as gel retardation) (Wilson et al. (2001) Journal of Bacteriology, 183: 2151-2155). The effect of DNA-binding proteins on the expression of other genes can be detected by various reporter gene assay methods which have been thoroughly described (Sambrook et al., Molecular Cloning: a laboratory manual, 2^{nd} Ed. Cold Spring Harbor Laboratory Press, Cold Spring Harbor, N.Y. USA, 1989). The intracellular enzymatic activities can be determined by various described methods (Donahue et al. (2000) Journal of Bacteriology 182 (19): 5624-5627; Ray et al. (2000) Journal of Bacteriology 182 (8): 2277-2284; Freedberg et al. (1973) Journal of Bacteriology 115 (3): 816-823). If no specific methods for determining the activity of a particular enzyme are indicated in the following statements, the determination of the increase in the enzymatic activity and also the determination of the reduction in an enzymatic activity preferably takes place by means of the methods described in Hermann et al. (Electrophoresis 22: 1712-1723 (2001)), Lohaus et al. (Biospektrum 5: 32-39 (1998)), Lottspeich (Angewandte Chemie 111: 2630-2647 (1999)) and Wilson et al. (Journal of Bacteriology 183: 2151-2155 (2001)).

If the increase in the enzymatic activity is brought about by mutation of the endogenous gene, such mutations can be generated either randomly by classical methods, such as, for instance, by UV irradiation or by mutagenic chemicals, or specifically by means of methods of genetic manipulations such as deletion(s), insertion(s) and/or nucleotide exchange(s). These mutations result in genetically modified cells. Particularly preferred mutants of enzymes are in particular also those enzymes no longer subject, or at least less subject by comparison with the wild-type enzyme, to feedback inhibition.

If the increase in enzymatic activity is brought about by increasing the expression of an enzyme, then for example the copy number of the corresponding genes is increased, or the promoter region and regulatory region or the ribosome binding site located upstream from the structural gene is mutated. Expression cassettes incorporated upstream of the structural gene operate in the same way. It is additionally possible by inducible promoters to increase the expression at any desired time. A further possibility is, however, also to assign so-called enhancers as regulatory sequences to the enzyme gene, which likewise bring about increased gene expression via an improved interaction between RNA polymerase and DNA. Expression is likewise improved by measures to extend the lifetime of the m-RNA. The enzymatic activity is likewise enhanced moreover by preventing degradation of the enzyme protein. The genes or gene constructs are in this case either present in plasmids with differing copy number or are integrated and amplified in the chromosome. A further alternative possibility is to achieve overexpression of the relevant genes by modifying the composition of media and management of the culture. The skilled worker will find instructions for this inter alia in Martin et al. (Bio/Technology 5, 137-146 (1987)), in Guerrero et al. (Gene 138: 35-41 (1994)), Tsuchiya and Morinaga (Bio/Technology 6: 428-430 (1988)), in Eikmanns et al. (Gene 102: 93-98 (1991)), in EP-A 0 472 869, in U.S. Pat. No. 4,601,893, in Schwarzer and Puhler (Bio/Technology 9: 84-87 (1991)), in Reinscheid et al. (Applied and Environmental Microbiology 60: 126-132 (1994)), in LaBarre et al. (Journal of Bacteriology 175: 1001-1007 (1993)), in WO-A 96/15246, in Malumbres et al. (Gene 134: 15-24 (1993)), in JP-A-10-229891, in Jensen and Hammer (Biotechnology and Bioengineering 58: 191-195 (1998)) and in well-known textbooks of genetics and molecular biology. The measures described above lead, just like the mutations, to genetically modified cells.

Expression of the particular genes is increased for example by employing episomal plasmids. Suitable plasmids are in particular those which are replicated in coryneform bacteria. Numerous well-known plasmid vectors such as, for example, pZ1 (Menkel et al., Applied and Environmental Microbiology 64: 549-554 (1989)), pEKEx1 (Eikmanns et al., Gene 107: 69-74 (1991)) or pHS2-1 (Sonnen et al., Gene 107: 69-74 (1991)) are based on the cryptic plasmids pHM1519, pBL1 or pGA1. Other plasmid vectors, such as, for example, those based on pCG4 (U.S. Pat. No. 4,489,160) or pNG2 (Serwold-Davis et al., FEMS Microbiology Letters 66: 119-124 (1990)) or pAG1 (U.S. Pat. No. 5,158,891) can be employed in the same way.

Also suitable in addition are those plasmid vectors which can be used to apply the method of gene amplification by integration into the chromosome, as has been described for example by Reinscheid et al. (Applied and Environmental Microbiology 60: 126-132 (1994)) for duplication or amplification of the hom-thrB operon. In this method, the complete gene is cloned into a plasmid vector which can be replicated in a host (typically Escherichia coli), but not in Corynebacterium glutamicum. Suitable vectors are for example pSUP301 (Simon et al., Bio/Technology 1: 784-791 (1983)), pK18mob or pK19mob (Schäfer et al., Gene 145: 69-73 (1994)), PGEM-T (Promega Corporation, Madison, Wis., USA), pCR2.1-TOPO (Shuman, Journal of Biological Chemistry 269: 32678-84 (1994)), pCR®Blunt (Invitrogen, Groningen, Netherlands), pEM1 (Schrumpf et al., Journal of Bacteriology 173: 4510-4516)) or pBGS8 (Spratt et al., Gene 41: 337-342 (1986)). The plasmid vector which comprises the gene to be amplified is subsequently transferred by conjugation or transformation into the desired strain of Corynebacterium glutamicum. The method of conjugation is described for example by Schäfer et al., Applied and Environmental Microbiology 60: 756-759 (1994). Methods for transformation are described for example by Thierbach et al. (Applied Microbiology and Biotechnology 29: 356-362 (1988)), Dunican and Shivnan (Bio/Technology 7: 1067-1070 (1989)) and Tauch et al. (FEMS Microbiology Letters 123: 343-347 (1994)). Following homologous recombination by a crossover event, the resulting strain comprises at least two copies of the relevant gene.

The cells of the invention are preferably genetically modified cells. These may be prokaryotes or eukaryotes. They may moreover be mammalian cells (such as, for instance, human cells), plant cells or microorganisms such as yeast cells, fungi or bacterial cells, with particular preference for microorganisms and most preference for bacterial cells and yeast cells.

Bacterial, yeast and fungal cells suitable according to the invention are all those bacterial, yeast and fungal cells which are deposited at the Deutsche Sammlung von Mikroorganismen und Zellkulturen GmbH (DSMZ), Brunswick, Germany, as wild-type bacterial strains. Suitable bacterial cells belong to the genera which are listed under
http://www.dsmz.de/species/bacteria.htm.

Yeast cells suitable according to the invention belong to those genera which are listed under
http://www.dsmz.de/species/yeasts.htm
   and fungi suitable according to the invention are those listed under

http://www.dsmz.de/species/fungi.htm.

Cells particularly preferred according to the invention are those of the genera Corynebacterium, Brevibacterium, Bacillus, Lactobacillus, Lactococcus, Candida, Pichia, Kluveromyces, Saccharomyces, Bacillus, Escherichia and Clostridium, with particular preference for Bacillus flavum, Bacillus lactofermentum, Escherichia coli, Saccharomyces cerevisiae, Kluveromyces lactis, Candida blankii, Candida rugosa, Corynebacterium glutamicum, Corynebacterium efficiens and Pichia postoris, and most preference for Corynebacterium glutamicum.

The enzyme E₁ₐ is preferably a carboxylase, particularly preferably a pyruvate carboxylase (EC number 6.4.1.1) which catalyzes the conversion of pyruvate into oxaloacetate. Genes for pyruvate carboxylases (pyc genes) for example from Rhizobium etli (Dunn et al., J. Bacteriol. 178: 5960-5970 (1996), see also WO-A 99/53035), Bacillus subtillis (Genbank Accession No. Z97025), Mycobacterium tuberculosis (Genbank Accession No. Z83018), Pseudomonas fluorescens (WO-A-99/53035) and from Methanobacterium thermoautotrophicum (Mukhopadhyay, J. Biol. Chem. 273: 5155-5166 (1998)) have been cloned and sequenced. In addition, pyruvate carboxylase activity has been detected in Brevibacterium lactofermentum (Tosaka et al., Agric. Biol. Chem. 43: 1513-1519 (1979)) and in Corynebacterium glutamicum (Peters-Wendisch et al., Microbiology 143: 1095-1103 (1997)). The nucleotide sequence of the pyc gene is also described in DE-A 100 31 999, DE-A-198 31 609, U.S. Pat. No. 6,171,833, U.S. Pat. No. 6,403,351 and U.S. Pat. No. 6,455,284. Pyruvate carboxylases preferred according to the invention are those pyruvate carboxylates encoded by genes selected from the group including PC, Pcx, CG1516, CG1516, pyc-1, PYC2, AAR162Cp, pyr1, accC-2, pycA, pycA2, pca, Cg10689, pyc, pycB, accc, oadA, acc and accC1, with particular preference for the pyc gene. Pyruvate carboxylases preferred according to the invention are described in particular also in U.S. Pat. No. 6,455,284, U.S. Pat. No. 6,171,833, U.S. Pat. No. 6,884,606, U.S. Pat. No. 6,403,351, U.S. Pat. No. 6,852,516 and U.S. Pat. No. 6,861,246. However, it is possible in principle to use pyc genes of any conceivable origin, irrespective of whether they are from bacteria, yeasts, animals, fungi or plants. It is further possible to use all alleles of the pyc gene, especially including those arising from the degeneracy of the genetic code or by functionally neutral sense mutations.

A pyruvate carboxylase which is particularly preferred according to the invention is the mutant which is described in “A novel methodology employing Corynebacterium glutamicum genome information to generate a new L-lysine-producing mutant” (Onishi et al., Applied Microbiology and Biotechnology 58(2): 217-223 (2002)). In this mutation, the amino acid proline at position 458 was replaced by serine. The disclosure of this publication in relation to the possibility of producing pyruvate carboxylase mutants is hereby introduced as reference and forms part of the disclosure of the present invention. Cells particularly preferred according to the invention are accordingly those having the enzyme mutant described above as exogenous protein, and those having exogenous DNA sequences which code for such an enzyme and which express this enzyme in adequate quantity.

The production of cells with increased pyruvate carboxylase activity is described in detail inter alia in DE-A 100 31 999 and also in DE-A 198 31 609. The disclosure of these publications in relation to the different possibilities for increasing the activity of pyruvate carboxylase in cells, especially in bacteria of the genus Corynebacterium, is hereby likewise introduced as reference and forms part of the disclosure of the present invention.

The intracellular activity of the pyruvate carboxylase is preferably determined by the method described in the thesis by Petra Peters-Wendisch at the Forschungszentrum Jülich GmbH “Anaplerotische Reaktionen in Corynebacterium glutamicum: Untersuchung zur Bedeutung der PEP-Carboxylase und der Pyruvat-Carboxylase im Zentralstoffwechsel und bei der Aminosäureproduktion” (1996).

The enzyme Elb is preferably a carboxylase, particularly preferably a phosphoenolpyruvate carboxylase (EC 4.1.1.31), which catalyzes the conversion of phosphoenolpyruvate to oxaloacetate. Phosphoenolpyruvate carboxylases which are preferred according to the invention are those phosphoenolpyruvate carboxylases which are encoded by the genes selected from the group including F12M16.21, F14N22.13, K15M2.8, ppc, clpA, pepc, capP, Cg11585 and pepC, with particular preference for the ppc gene. ppc genes for wild-type phosphoenolpyruvate carboxylases or mutants of these enzymes are disclosed for example in U.S. Pat. No. 6,599,732, U.S. Pat. No. 5,573,945, U.S. Pat. No. 4,757,009 and in U.S. Pat. No. 4,980,285. The production of cells having increased phosphoenolpyruvate carboxylase activity is described inter alia in U.S. Pat. No. 4,757,009. The disclosure of this publication in relation to the procedure for the overexpression of phosphoenolpyruvate carboxylase in microorganisms is hereby likewise introduced as reference and forms part of the disclosure of the present invention. Besides overexpression of the enzyme, it is also possible to apply the other measures mentioned in connection with the enzyme E₁ₐ for increasing the enzymatic activity of the phosphoenolpyruvate carboxylase, inter alia including the expression of enzyme mutants. Mutants particularly preferred in this connection are those which do not require activation by acetyl-CoA and/or which are feedback inhibited in relation to aspartic acid (in this connection, see in particular U.S. Pat. No. 6,919,190). It is also possible in principle in connection with the phosphoenolpyruvate carboxylase to use the corresponding genes of any conceivable origin, irrespective of whether they are from bacteria, yeasts, plants, animals or fungi. It is further possible also in this case to use all alleles of the ppc gene, especially including those arising from the degeneracy of the genetic code or by functionally neutral sense mutations.

The activity of the phosphoenolpyruvate carboxylase is preferably determined by the method described in the thesis by Petra Peters-Wendisch at the Forschungszentrum Jülich GmbH “Anaplerotische Reaktionen in Corynebacterium glutamicum: Untersuchung zur Bedeutung der PEP-Carboxylase und der Pyruvat-Carboxylase im Zentralstoffwechsel und bei der Aminosäureproduktion” (1996).

A survey of phosphoenolpyruvate carboxylases and pyruvate carboxylases is given in particular also by Sauer and Eikmanns (FEMS Microbiology Reviews 29: 765-794 (2005)).

The enzyme E₂ is preferably a decarboxylase, particularly preferably a glutamate decarboxylase or an aspartate decarboxylase, with most preference for a 1-aspartate 1-decarboxylase (EC number 4.1.1.11) which is encoded by the panD gene. The aspartate decarboxylase catalyzes the conversion of aspartate to beta-alanine. Genes for the aspartate decarboxylase (panD genes) inter alia from Escherichia coli (FEMS Microbiology Letters 143: 247-252 (1996)), Photorhabdus luminescens subsp. laumondii, Mycobacterium bovis subsp. bovis, and from many other microorganisms have already been cloned and sequenced. In particular, the nucleotide sequence of the panD gene from Corynebacterium glutamicum is described in DE-A-198 55 313. It is possible in principle to use panD genes of any conceivable origin, irrespective of whether they are from bacteria, yeasts, plants, animals or fungi. It is further possible to use all alleles of the panD gene, especially including those arising from the degeneracy of the genetic code or through the functionally neutral sense mutations.

An aspartate decarboxylase which is particularly preferred according to the invention besides the aspartate decarboxylase from Corynebacterium glutamicum is the Escherichia coli mutant DV9 (Vallari and Rock, Journal of Bacteriology 164: 136-142 (1985)). The disclosure of this publication in relation to the aforementioned mutant is hereby introduced as reference and forms part of the disclosure of the present invention.

The production of cells with increased aspartate decarboxylase activity is described in detail inter alia in DE-A 198 55 314. The disclosure of these publications in relation to the different possible ways of increasing the activity of aspartate decarboxylase in cells, especially in bacteria of the genus Corynebacterium, is hereby likewise introduced as reference and forms part of the disclosure of the present invention. Cells which are particularly preferred according to the invention are those which have enzyme mutant DV9 described above from Escherichia coli or else panD from Corynebacterium glutamicum, and those which have DNA sequences which code for one of these enzymes and which express this enzyme in sufficient quantity.

The aspartate decarboxylase activity is determined by the assay method described by Dusch et al. (Applied and Environmental Microbiology 65(4): 1530-1539 (1999)) in the section entitled “Aspartate decarboxylase activity assay”.

If the cell of the invention is a genetically modified Corynebacterium glutamicum cell, it may be sufficient for only the activity of the enzyme E₂ to be increased, because the wild type of these cells already has a comparatively high pyruvate carboxylase activity.

Nevertheless, even when using Corynebacterium glutamicum it is preferred for both the activity of the enzyme E₁ₐ and the activity of the enzyme E₂, or both the activity of the enzyme E_{1b} and the activity of the enzyme E₂, in the cell of the invention to be increased.

The cells of the invention are further characterized in that, besides properties a) or b), preferably a) and b), it is characterized by at least one of the properties c) or d):
a) the genetically modified cell is able to export beta-alanine out of the cell;
b) the genetically modified cell is able to convert beta-alanine into 3-hydroxypropionic acid.

The term “3-hydroxypropionic acid” as used herein includes the protonated form of 3-hydroxypropionic acid as well as the deprotonated form of 3-hydroxypropionic acid (=3-hydroxypropionate) and mixtures of protonated and deprotonated form. The term “export” includes both the active and the passive transport of beta-alanine out of the cell into the medium surrounding the cell.

Cells of the invention which satisfy condition c) are preferably characterized in that they have endogenous and/or exogenous, preferably exogenous, transport enzymes in the cell membrane which are able to transport, selectively or nonselectively, preferably selectively, actively or passively, where appropriate in exchange for or together with ions such as sodium, potassium or chlorine, beta-alanine through the cell membrane to the outside, that is to say into the region outside the cell (=efflux of the beta-alanine out of the cell). It is particularly preferred in this connection according to the invention for the genetically modified cell to exhibit an efflux of beta-alanine out of the cell which is increased by comparison with its wild type, preferably increased by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%. An efflux which is increased by 10% means in this connection that the genetically modified cell is able to export 10% more beta-alanine out of the cell by comparison with its wild type under identical conditions, in particular with an indentical intracellular and extracellular beta-alanine concentration, in a defined time interval. The increased efflux is preferably achieved by increasing the activity of the aforementioned transport enzymes, it being possible for the increase in turn to be effected by the techniques already mentioned in connection with the enzymes E₁ₐ, E_{1b} and E₂ (mutation of the transport enzyme or increase in the transport enzyme gene expression).

Transport enzymes preferred in this connection are, for example, the so-called multi-drug resistance proteins (MDR proteins), for example with the genes ebrA and ebrB, and the so-called multi-drug efflux transporters, with particular preference for the multi-drug efflux transporters for example having the blt and bmr genes. Suitable transport systems for beta-alanine are also described in “Handbook of Corynebacterium glutamicum”, L. Eggeling and M. Bott, editors, CRC Press, Boca Raton, USA, 2005, Chapter IV, “Genomic Analyses of Transporter Proteins in Corynebacterium glutamicum and Corynebactenium efficiens”, B. Winnen, J. Felce, and M. H. Saier, Jr., pages 149-186. Further suitable transport systems for beta-alanine, especially those encoded by the cycA gene, are described in Schneider et al (Appl. Microbiol. Biotechnol. 65(5): 576-582 (2004)). Suitable transport systems for beta-alanine are further described in Anderson and Thwaites (J. Cell. Physiol. 204(2): 604-613 (2005)), Brechtel and King (Biochem. J. 333: 565-571 (1998)), Guimbal et al. (Eur. J. Biochem. 234(3): 794-800 (1995)), Munck and Munck (Biochim. Biophys. Acta 1235(1):93-99 (1995)) and Shuttleworth and Goldstein (J. Exp. Zool. 231(1): 39-44 (1984)).

Cells of the invention which satisfy condition d) are able to convert the beta-alanine formed into 3-hydroxypropionic acid. At least two variants are conceivable in this connection.

In variant A, the cells can convert the beta-alanine via beta-alanyl-CoA, acrylyl-CoA and hydroxypropionyl-CoA into 3-hydroxypropionic acid. It is particularly preferred in this connection for the cell to exhibit an activity which is increased by comparison with its wild type, preferably increased by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of at least one, preferably all, of the following enzymes E₃ to E₆:
of an enzyme E₃ which catalyzes the conversion of beta-alanine into beta-alanyl-coenzyme A,
of an enzyme E₄ which catalyzes the conversion of beta-alanyl-coenzyme A into acrylyl-coenzyme A,
of an enzyme E₅ which catalyzes the conversion of acrylyl-coenzyme A into 3-hydroxypropionyl-coenzyme A,
of an enzyme E₆ which catalyzes the conversion of 3-hydroxypropionyl-coenzyme A into 3-hydroxy-propionic acid.

Genetically modified cells which are particularly preferred according to the invention are in this connection those in which, where appropriate in addition to the increase in at least one of the enzymatic activities E₁ₐ or E_{1b}, and E₂, the activity of the following enzymes or enzyme combinations is increased: E₃, E₄, E₅, E₆, E₃E₄, E₃E₅, E₃E₆, E₄E₅, E₄E₆, E₅E₆, E₃E₄E₅, E₃E₄E₆, E₃E₅E₅, E₄E₅E₆ or E₃E₄E₅E₆.

The increase in the enzymatic activity of enzymes E₃ to E₆ can also in this case be effected by the techniques mentioned in connection with the enzymes E₁ₐ, E₁ₐ and E₂, such as mutation or increasing enzymatic expression.

It is further preferred in this connection for the enzyme
E₃ to be a coenzyme A transferase (EC 2.8.3.1) or coenzyme A synthetase, preferably a coenzyme A transferase,
E₄ to be a beta-alanyl-coenzyme A ammonium-lyase (EC 4.3.1.6),
E₅ to be a 3-hydroxypropionyl-coenzyme A dehydratase (EC 4.2.1.-, in particular EC 4.2.1.17) and
E₆ to be a coenzyme A transferase (EC 2.8.3.1), 3-hydroxypropionyl-coenzyme A hydrolase (EC 3.1.2.-) or 3-hydroxybutyryl-coenzyme A hydrolase (EC 3.1.2.4), preferably a coenzyme A transferase.

Preferred enzymes having a CoA transferase activity are those from Megasphaera elsdenii, Clostridium propionicum, Clostridium kluyveri and also from Escherichia coli. Examples which may be mentioned of a DNA sequence encoding a CoA transferase at this point are the sequence, designated SEQ ID NO: 24 from Megasphaera elsdenii in WO-A 03/062173. Further preferred enzymes are those variants of CoA transferase described in WO-A 03/062173.

Suitable enzymes having a beta-alanyl-coenzyme A ammonium-lyase activity are for example those from Clostridium propionicum. DNA sequences which code for such an enzyme can be obtained for example from Clostridium propionicum as described in Example 10 of WO-A 03/062173. The DNA sequence which codes for the beta-alanyl-coenzyme A ammonium-lyase from Clostridium propionicum is indicated in WO-A 03/062173 as SEQ ID NO: 22.

Suitable enzymes having a 3-hydroxypropionyl-coenzyme A dehydratase activity are especially those enzymes encoded by genes selected from the group including ECHS1, EHHADH, HADHA, CG4389, CG6543, CG6984, CG8778, ech-1, ech-2, ech-3, ech-5, ech-6, ech-7, FCAALL.314, FCAALL.21, FOX2, ECI12, ECI1, paaF, paaG, yfcx, fadB, faoA, fadBlx, phaB, echA9, echA17, fad-1, fad-2, fad-3, paaB, echA7, dcaE, hcaA, RSp0671, RSp0035, RSp0648, RSp0647, RS03234, RS03271, RS04421, RS04419, RS02820, RS02946, paaG2, paaG1, ech, badK, crt, ydbS, eccH2, pimF, paaG3, fabJ-1, caiD-2, fabJ-2, ysiB, yngF, yusL, phaA, phaB, fucA, caiD, ysiB, echA3, echA5, echA6, echA7, echA8, echA14, echA15, echA16, echA17, echA18.1, echA19, echA20, echA21, echA2, echA4, echA9, echA11, echA10, echA12, echA13, echA18, echA1, fadB-1, echA8-1, echA12-2, fadB-2, echA16-2, Cg10919, fadB1, SCF41.23, SCD10.16, SCK13.22, SCP8.07c, StBAC16H6.14, SC5F2A.15, SC6A5.38, faoA, hbd1, crt, hbd-1, hbd-2, hbd-5, fad-4, hbd-10, fad-5, hbd1, paaF-1, paaF-2, paaF-3, paaF-4, paaF-5, paaF-6 and paaF-7. Examples of 3-hydroxypropionyl-coenzyme A dehydratases suitable according to the invention which may be mentioned are in particular those from Chloroflexus aurantiacus, Candida rugosa, Rhodosprillium rubrum and Rhodobacter capsulates. A particular example of a DNA sequence coding for a 3-hydroxypropionyl-coenzyme A dehydratase is indicated for example in WO-A 02/42418 as SEQ ID NO: 40.

The production of genetically modified cells in which at least one, preferably all, of the aforementioned enzymatic activities E₃ to E₆ has or have been increased is described for example in the examples of WO-A 02/42418 and of WO-A 03/062173. The disclosure of these two publications in relation to increasing the activities of these enzymes in cells is hereby introduced as reference and forms part of the disclosure of the present invention.

In variant B, the cells can convert the beta-alanine via malonic semialdehyde into 3-hydroxypropionic acid. It is particularly preferred in this connection for the cell to exhibit an activity which is increased by comparison with its wild type, preferably increased by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of at least one, preferably both, of the enzymes E7 and E8:
of an enzyme E₇ which catalyzes the conversion of beta-alanine into malonic semialdehyde,
of an enzyme E₈ which catalyzes the conversion of malonic semialdehyde into 3-hydroxypropionic acid.

Genetically modified cells which are particularly preferred according to the invention in this connection are those in which, where appropriate in addition to increasing at least one of the enzymatic activities E₁ₐ or E_{1b} and E₂, the activity of the following enzymes or enzyme combinations is increased: E₇, E₈ and E₇E₈. The increase in the enzymatic activity of enzymes E₇ and E₈ can also in this case be effected via the techniques mentioned in connection with the enzyme E₁ₐ, E_{1b} and E₂, such as mutation or increasing enzymatic expression.

It is further preferred in this connection for the enzyme
E₇ to be a beta-alanine-2-oxoglutarate amino-transferase (EC 2.6.1.19) or a taurine-2-oxoglutarate transaminase (2.6.1.55), preferably a beta-alanine-2-oxoglutarate aminotransferase, and
E₈ to be a 3-hydroxypropionate dehydrogenase (EC 1.1.1.59) or 3-hydroxybutyrate dehydrogenase (EC 1.1.1.30), but preferably a 3-hydroxy-propionate dehydrogenase (EC 1.1.1.59).

The beta-alanine-2-oxoglutarate aminotransferase genes are known for example from Neurospora crassa (“Uber die beta-Alanin-alpha-Ketoglutarat-Transaminase”, Aurich and Hoppe-Seyler's, Z. Physiol. Chem. 326: 25-33 (1961)). Further information about genes of this enzyme from other microorganisms can be taken in particular from the KEGG GENE database (KEGG=Kyoto Encyclopedia of Genes and Genomes). The genes of 3-hydroxypropionate dehydrogenase or of 3-hydroxybutyrate dehydrogenase from a wide variety of microorganisms can also be taken from the KEGG GENE database.

If the cells of the invention satisfy condition d), it is further preferred for the cell to exhibit a beta-alanine efflux which is reduced by comparison with its wild type, preferably reduced by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%. An efflux which is reduced by 10% means in this connection that the genetically modified cell is able to export 10% less beta-alanine from the cell by comparison with its wild type under identical conditions, in particular with identical intracellular and extracellular beta-alanine concentration, in a defined time interval. The reduced efflux is preferably achieved by reducing the activity of the aforementioned transport enzymes, it being possible for the reduction to be effected by mutation of the transport enzyme or reduction of the transport enzyme gene expression. It may also be advantageous to employ as cells which satisfy condition d) those cells whose wild type is unable to export beta-alanine out of the cell.

The cells of the invention are alone (if condition d) is satisfied) or else in combination with other microorganisms, which are able to produce 3-hydroxypropionic acid from beta-alanine (if condition c) is satisfied), able to form 3-hydroxypropionic acid from pyruvate.

Beginning with pyruvate as starting point for the production of beta-alanine, in turn two different embodiments of the cells of the invention are now conceivable.

In the first embodiment of the cells of the invention, they are able to produce the pyruvate required to produce beta-alanine, in particular also from glycerol as carbon source.

It is particularly preferred in this connection for the cell of the invention to exhibit an activity which is increased by comparison with its wild type, preferably increased by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of at least one, preferably all, of the following enzymes E₉ to E₂₂:
of an enzyme E₉ which facilitates the diffusion of glycerol into the cell,
of an enzyme E₁o which catalyzes the conversion of glycerol into glycerol 3-phosphate,
of an enzyme E₁l which catalyzes the conversion of glycerol 3-phosphate into dihydroxyacetone phosphate,
of an enzyme E₁₂ which catalyzes the transfer of sulfur to the sulfur acceptor thioredoxin 1,
of an enzyme E₁₃ which catalyzes the hydrolysis of phospholipids to form alcohols and glycerol,
of an enzyme E₁₄ which catalyzes the transport of glycerol 3-phosphate into the cell in exchange for phosphate;
of an enzyme E₁₅ which catalyzes the conversion of dihydroxyacetone phosphate into glyceraldehyde 3-phosphate,
of an enzyme E₁₆ which catalyzes the conversion of glyceraldehyde 3-phosphate into 1,3-biphospho-glycerate,
of an enzyme E₁₇ which catalyzes the conversion of 1,3-biphosphoglycerate into 3-phosphoglycerate,
of an enzyme E₁₈ which catalyzes the conversion of 3-phosphoglycerate into 2-phosphoglycerate,
of an enzyme E₁₉ which catalyzes the conversion of 2-phosphoglycerate into phosphoenolpyruvate,
of an enzyme E₂₀ which catalyzes the conversion of phosphoenolpyruvate into pyruvate,
of an enzyme E₂₁ which catalyzes the conversion of glycerol into dihydroxyacetone,
of an enzyme E₂₂ which catalyzes the conversion of dihydroxyacetone into dihydroxyacetone phosphate.

Genetically modified cells which are particularly preferred according to the invention in this connection are those in which, where appropriate in addition to the increase in one or more of the enzymatic activities E₁ₐ or E_{1b} and E₂, and where appropriate one or more of the enzymatic activities E₃ to E₆ or E₇ and E₈, the activity of the following enzymes or enzyme combinations is increased: E₉, E₁₀, E₁₁, E₁₂, E₁₃, E₁₄, E₁₅ , E₁₆ , E₁₇ , E₁₈, E₁₉, E₂₀, E₂₁ , E₂₂, E₁₀E₁₁, with particular preference for an increase in one or more of the enzymatic activities selected from the group consisting of E₉, E₁₀, E₁₁, E₁₃, E₁₄, E₂₁ and E₂₂, and with most preference for an increase in the enzymatic activities E₁₀ and E₁₁.

It is particularly preferred in this connection for the enzyme
E₉ to be an aquaglyceroporin (glycerol facilitator) preferably encoded by the glpF gene,
E₁₀ to be a glycerol kinase (EC 2.7.1.30) preferably encoded by the glpK gene,
E₁₁, to be a glycerol-3-phosphate dehydrogenase (EC 1.1.99.5), preferably an FAD-dependent glycerol-3-phosphate dehydrogenase, where the glycerol-3-phosphate dehydrogenase is preferably encoded by the glpA gene, the glpB gene, the glpC gene or the glpD gene, particularly preferably the glpD gene,
E₁₂ to be a sulfur transferase encoded by the glpE gene,
E₁₃ to be a glycerol phosphodiesterase (EC 3.1.4.46), preferably encoded by the glpQ gene,
E₁₄ to be a glycerol-3-phosphate permease preferably encoded by the glpT gene
E₁₅ to be a triose-phosphate isomerase (EC 5.3.1.1),
E₁₆ to be a glyceraldehyde-3-phosphate dehydrogenase (EC 1.2.1.12),
E₁₇ to be a phosphoglycerate kinase (EC 2.7.2.3),
E₁₈ to be a phosphoglycerate mutase (EC 5.4.2.1),
E₁₉ to be an enolase (EC 4.2.1.11),
E₂₀ to be a pyruvate kinase (EC 2.7.1.40),
E₂₁ to be a glycerol dehydrogenase (EC 1.1.1.6) preferably encoded by the gldA gene, and
E₂₂ to be a dihydroxyacetone kinase (EC 2.7.1.29) preferably encoded by the dhaK gene.

The gene sequences of the aforementioned enzymes are disclosed in the literature and can be taken for example from the KEGG GENE database, the databases of the National Center for Biotechnology Information (NCBI) of the National Library of Medicine (Bethesda, Md., USA) or the nucleotide sequence database of the European Molecular Biologies Laboratories (EMBL, Heidelberg, Germany or Cambridge, UK). In addition, the gap gene encoding glyceraldehyde-3-phosphate dehydrogenase (Eikmanns (1992), Journal of Bacteriology 174: 6076-6086), the tpi gene coding for the triose-phosphate isomerase (Eikmanns (1992), Journal of Bacteriology 174: 6076-6086), and the pgk gene coding for the 3-phosphoglycerate kinase (Eikmanns (1992), Journal of Bacteriology 174: 6076-6086) are disclosed in other sources.

It is possible with the known genes of the enzymes E₉ to E₂₂ to produce genetically modified cells in which at least one, particularly preferably at least two, further preferably at least three and most preferably all of the activities of the enzymes E₉ to E₂₂ has been increased via the techniques described above in connection with the enzyme E₁ₐ, E_{1b} and E₂ (mutation of the enzyme or increasing enzymatic expression). These cells are able to be cultured in the presence of glycerol as sole carbon source (or else together with carbohydrates as further carbon source).

Besides the increase in one or more of the enzymatic activities E₉ to E₂₂, it may also be advantageous in this connection if the following genes express, preferably heterologously, in the cells of the invention:
the glpG gene or the b3424 gene,
the glpx gene or the 3925 gene,
the dhaR gene, the ycgu gene or the b1201 gene
the fsa gene, the mipB gene, the ybiz gene or the B0825 gene
the talC gene, the fsaB gene, the yijG gene or the b3946 gene.

The nucleotide sequences of these genes can in turn be taken from the KEGG GENE database, the databases of the National Center for Biotechnology Information (NCBI) of the National Library of Medicine (Bethesda, Md., USA) or the nucleotide sequence database of the European Molecular Biologies Laboratories (EMBL, Heidelberg, Germany or Cambridge, UK).

In the second embodiment of the cells of the invention, they are able to obtain the pyruvate, required to produce the beta-alanine, from glycerol at least only to a small extent or not at all. In this case, the provision of pyruvate in the cells takes place principally through glycolysis. Cells of this type can be cultured in a nutrient medium which contains carboyhydrates such as, for example, glucose as carbon source.

It is preferred in this case for the cells of the invention, where appropriate besides an increased activity of at least one, preferably all, of the aforementioned enzymes E₉ to E₂₂, to exhibit an activity which is increased by comparison with their wild type, preferably increased by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of at least one, preferably all, of the following enzymes E₁₆ to E₂₀ and E₂₃ to E₂₇:
of an enzyme E₂₃ which facilitates the diffusion of glycerol into the cell,
of an enzyme E₂₄ which catalyzes the conversion of glucose into α-D-glucose 6-phosphate,
of an enzyme E₂₅ which catalyzes the conversion of α-D-glucose 6-phosphate into β-D-fructose 6-phosphate,
of an enzyme E₂₆ which catalyzes the conversion of β-D-fructose 6-phosphate into β-D-fructose 1,6-biphosphate,
of an enzyme E₂₇ which catalyzes the conversion of β-D-fructose 1,6-biphosphate into glyceraldehyde 3-phosphate and dihydroxyacetone phosphate,
of an enzyme E₁₆ which catalyzes the conversion of glyceraldehyde 3-phosphate into 1,3-biphospho-glycerate,
of an enzyme E₁₇ which catalyzes the conversion of 1,3-biphosphoglycerate into 3-phosphoglycerate,
of an enzyme E₁₈ which catalyzes the conversion of 3-phosphoglycerate into 2-phosphoglycerate,
of an enzyme E₁₉ which catalyzes the conversion of 2-phosphoglycerate into phosphoenolpyruvate, and
of an enzyme E₂₀ which catalyzes the conversion of phosphoenolpyruvate into pyruvate.

Genetically modified cells which are particularly preferred according to the invention are in this connection those in which, where appropriate in addition to the increase in one or more of the enzymatic activities E₁ₐ or E_{1b} and E₂, and where appropriate one or more of the enzymatic activities E₃ to E₆ or E₇ and E₈, the activity of the following enzymes or enzyme combinations is increased: E₁₆, E₁₇, E₁₈, E₁₉, E₂₀, E₂₃, E₂₄, E₂₅ D₂₆, E₂₇ and E₁₆E₁₇E₁₈E₁₉E₂₀E₂₃E₂₄E₂₅E₂₆E₂₇.

It is particularly preferred in this connection for the enzyme
E₂₃ to be a glucose transporter, preferably a glucose transporter encoded by a gene selected from the group comprising glut1, gluP or fucp, or a glucose permease (2.7.1.69),
E₂₄ to be a glucokinase (2.7.1.2),
E₂₅ to be a glucose-6-phosphate isomerase (EC 5.3.1.9),
E₂₆ to be a 6-phosphofructokinase (EC 2.7.1.11),
E₂₇ to be a fructose-bisphosphate aldolase (EC 4.1.2.13),
E₁₆ to be a glyceraldehyde-3-phosphate dehydrogenase (EC 1.2.1.12),
E₁₇ to be a phosphoglycerate kinase (EC 2.7.2.3),
E₁₈ to be a phosphoglycerate mutase (EC 5.4.2.1),
E₁₉ to be an enolase (EC 4.2.1.11), and
E₂₀ to be a pyruvate kinase (EC 2.7.1.40).

The nucleotide sequences of these genes can in turn be taken from the KEGG GENE database, the databases of the National Center for Biotechology Information (NCBI) of the National Library of Medicine (Bethesda, Md., USA), the nucleotide sequence database of the European Molecular Biologies Laboratories (EMBL, Heidelberg, Germany and Cambridge, UK).

It is further preferred in connection with this second embodiment of the cell of the invention for, besides the aforementioned enzymes E₁ₐ or E_{1b} and/or E₂, and where appropriate at least one of the enzymes E₃ to E₆, E₇ or E₈, and E₁₂ to E₂₇, the activity of enzymes of the phosphotransferase system has also been increased. It is particularly preferred in this connection for there to be enhanced, preferably overexpression of the ptsI and ptsM genes in the cells of the invention. In this connection, reference is made in particular to U.S. Pat. Nos. 6,680,187 and 6,818,432, whose disclosure in relation to the possible ways of overexpressing the ptsI and ptsM genes is hereby introduced as reference and forms part of the disclosure of the present invention.

It is further possible for the activity of aspartate aminotransferase (EC 2.6.1.1.A) to be increased in the cells of the invention, irrespective of whether they use glycerol or glucose as primary nutrient source and also irrespective of whether they form 3-hydroxy-propionic acid alone or in combination with other cells via a malonic semialdehyde or via beta-alanyl-coenzyme A, acrylyl-coenzyme A and 3-hydroxypropionyl-coenzyme A. The sequence of the corresponding gene (aspB) can be taken inter alia from the KEGG GENE database.

It is further preferred in the cells of the invention to diminish the activity of enzymes
E₂₈ which catalyze the conversion of oxaloacetate into phosphoenolpyruvate such as, for instance, phosphoenylpyruvate carboxykinase (EC 4.1.1.49) (see also DE-A 199 50 409),
E₂₉ which catalyze the conversion of pyruvate to acetate such as, for instance, pyruvate oxidase (EC 1.2.2.2) (see also DE-A 199 51 975),
E₃0 which catalyze the conversion of α-D-glucose 6-phosphate into β-D-fructose 6-phosphate (see also U.S. Ser. No. 09/396,478),
E₃₁ which catalyze the conversion of beta-alanine into carnosine such as, for instance, carnosine synthase (EC 6.3.2.11),
E₃₂ which catalyze the conversion of beta-alanine into alpha-alanine such as, for instance, alanine aminomutase,
E₃₃ which catalyze the conversion of beta-alanine into (R)-pantothenate such as, for instance, pantothenate-beta-alanine ligase (EC 6.3.2.1),
E₃₄ which catalyze the conversion of beta-alanine into N-carbomyl-beta-alanine such as, for instance, beta-ureidopropionase (EC 3.5.1.6),
E₃₅ which catalyze the conversion of pyruvate into lactate such as, for instance, 1-lactate dehydrogenase (EC 1.1.1.27) or lactate-malate transhydrogenase (EC 1.1.99.7),
E₃₆ which catalyze the conversion of pyruvate into acetyl-coenzyme A such as, for instance, pyruvate dehydrogenase (EC 1.2.1.51),
E₃₇ which catalyzes the conversion of pyruvate into acetyl phosphate such as, for instance, pyruvate oxidase (EC 1.2.3.3),
E₃₈ which catalyzes the conversion of pyruvate into acetate such as, for instance, pyruvate dehydrogenase (EC 1.2.2.2),
E₃₉ which catalyze the conversion of pyruvate into phosphoenolpyruvate such as, for instance, phosphoenolpyruvate synthase (EC 2.7.9.2) or pyruvate-phosphate dikinase (EC 2.7.9.1), and/or
E₄₀ which catalyze the conversion of pyruvate into alanine such as, for instance, alanine transaminase (2.6.1.2) or alanine-oxo-acid transaminase (EC 2.6.1.12),
   preferably by at least 10%, particularly preferably by at least 25%, further preferably by at least 50%, further even more preferably by at least 75%, further preferably by at least 100% and most preferably by at least 500%, maximally preferably up to a maximum of 5000%, particularly up to a maximum of 2500%, with particular preference for diminution of the enzymes E₂₈, E₃₀, E₃₃, E₃₅, E₃₆ and E₄₀.

The term “diminish” describes in this connection the reduction or elimination of the intracellular activity of one or more enzymes in a cell which are encoded by the appropriate DNA, for example by using a weak promoter or using a gene or allele which codes for a corresponding enzyme with a low activity, or in-activating the appropriate gene or enzyme and, where appropriate, combining these measures.

It may in a particular embodiment of the cells of the invention in particular be worthwhile to promote purposely the pentose phosphate pathway, for example by increasing the activity of glucose-6-phosphate dehydrogenase (EC 1.1.1.49) and of 6-phosphogluconate dehydrogenase (EC 1.1.1.44), and at the same time inhibiting glycolysis, for example by diminishing the activity of glucose-6-phosphate isomerase as described in WO-A 01/07626.

In a particular embodiment of the cell of the invention, the activity of glutamate dehydrogenase (EC 1.4.1.4) therein is increased by one of the techniques mentioned in connection with the enzyme E₁ₐ, E_{1b} and E₂. The genes of this enzyme from numerous microorganisms can likewise be taken from the KEGG GENE database. Furthermore, U.S. Pat. No. 6,355,454 and WO-A 00/53726 describe genes of glutamate dehydrogenase and possible ways of overexpressing this enzyme. The disclosure of these publications in relation to carrying out the overexpression of glutamate dehydrogenase in cells is hereby introduced as reference and forms part of the disclosure of the present invention.

It is further preferred with the genetically modified cells of the invention for them to form beta-alanine and alpha-alanine in the ratio of at least 2:1, particularly preferably at least 3:1 and further preferably at least 4:1 by weight. Formation of beta-alanine and alpha-alanine in the ratio of at least 2:1 by weight means in this connection that the cells form, preferably form and release into the nutrient medium surrounding the cells, at least twice as much beta-alanine as alpha-alanine within a time period of 29 hours at 37° C.

It is particularly preferred in this connection with the cells of the invention for
the activity of glutamate dehydrogenase (EC 1.4.1.4) to be increased, and
the activity of pyruvate carboxylase (EC 6.4.1.1) to be increased, and
the activity of aspartate decarboxylase (EC 4.1.1.11) to be increased, and
the activity of glucose-6-phosphate isomerase (EC 5.3.1.9) to be diminished.

The present invention relates in particular to a genetically modified cell which exhibits

an increased pyruvate carboxylase activity (EC 6.4.1.1), preferably through expression of the mutant described in “A novel methodology employing Corynebacterium glutamicum genome information to generate a new L-lysine-producing mutant” (Ohnishi et al., Applied Microbiology and Biotechnology 58: 217-223 (2002)), and
an increased aspartate decarboxylase activity (EC 4.1.1.11), preferably through an asparate decarboxylase from Corynebacterium glutamicum
and at least one of, preferably all the following properties:
increased coenzyme A transferase activity (EC 2.8.3.1),
increased beta-alanyl-coenzyme A ammonium-lyase activity (EC 4.3.1.6), and
increased 3-hydroxypropionyl-coenzyme A dehydratase activity (EC 4.2.1.- in particular EC 4.2.1.17).

The present invention further relates in particular to a genetically modified cell which exhibits
an increased phosphoenolpyruvate carboxylase activity (EC 4.1.1.31) and
an increased aspartate decarboxylase activity (EC 4.1.1.11), preferably through an asparate decarboxylase from Corynebacterium glutamicum
   and at least one of, preferably all the following properties:
an increased coenzyme A transferase activity (EC 2.8.3.1),
an increased beta-alanyl-coenzyme A ammonium-lyase activity (EC 4.3.1.6), and
an increased 3-hydroxypropionyl-coenzyme A dehydratase activity (EC 4.2.1.- in particular EC 4.2.1.17).

The present invention also relates to a genetically modified cell which exhibits
an increased pyruvate carboxylase activity (EC 6.4.1.1), preferably through expression of the mutant described in “A novel methodology employing Corynebacterium glutamicum genome information to generate a new L-lysine-producing mutant” (Ohnishi et al., Applied Microbiology and Biotechnology 58: 217-223 (2002)), and
an increased aspartate decarboxylase activity (EC 4.1.1.11), preferably through an asparate decarboxylase from Corynebacterium glutamicum
   and at least one of, preferably all the following properties:
increased beta-alanine-2-oxoglutarate amino-transferase activity (EC 2.6.1.19), and
increased 3-hydroxypropionate dehydrogenase activity (EC 1.1.1.59).

The present invention further relates in particular to a genetically modified cell which exhibits
increased phosphoenolpyruvate carboxylase activity (EC 4.1.1.31) and
increased aspartate decarboxylase activity (EC 4.1.1.11), preferably through an asparate decarboxylase from Corynebacterium glutamicum
   and at least one of, preferably all the following properties:
increased beta-alanine-2-oxoglutarate amino-transferase activity (EC 2.6.1.19), and
increased 3-hydroxypropionate dehydrogenase activity (EC 1.1.1.59).

The present invention also relates to a genetically modified cell which exhibits
increased pyruvate carboxylase activity (EC 6.4.1.1), preferably through expression of the mutant described in “A novel methodology employing Corynebacterium glutamicum genome information to generate a new L-lysine-producing mutant” (Ohnishi et al., Applied Microbiology and Biotechnology 58: 217-223 (2002)), and
increased aspartate decarboxylase activity (EC 4.1.1.11), preferably through an asparate decarboxylase from Corynebacterium glutamicum
   and at least one of, preferably all the following properties:
increased glycerol-3-phosphate dehydrogenase activity (EC 1.1.99.5), preferably through a glycerol-3-phosphate dehydrogenase encoded by the glpD gene, and
increased glycerol kinase activity (EC 2.7.1.30), preferably through a glycerol kinase encoded by the glpK gene,
   where the cell in this case is preferably a microorganism of the strain Corynebacterium glutamicum.

A further contribution to achieving the objects mentioned at the outset is provided by methods for producing a genetically modified cell which is characterized by at least one of the properties C) or D):
A) the genetically modified cell is able to export beta-alanine out of the cell,
B) the genetically modified cell is able to convert beta-alanine into 3-hydroxypropionic acid,
   including at least one, preferably both, of steps A) and B) of the method:
C) increasing the activity of an enzyme E₁ₐ which catalyzes the conversion of pyruvate into oxaloacetate, or of an enzyme E_{1b} which catalyzes the conversion of phosphoenolpyruvate into oxaloacetate, in a cell, and
D) increasing the activity of an enzyme E₂ which catalyzes the conversion of aspartate into beta-alanine in a cell.

The enzymes E₁ₐ, E_{1b} and E₂ are preferably the enzymes previously described in connection with the cells of the invention. The increase in the aforementioned enzymatic activities is preferably effected by the genetic engineering methods described in connection with the cells of the invention, and also to the extent described in connection with the cells of the invention. The cells in which the activity of the enzymes E₁ₐ or E_{1b} and/or E₂ is increased and which are preferably employed are those genera and strains which have already been mentioned above in connection with the genetically modified cells of the invention.

Cells particularly preferably employed in the method of the invention are those of the genera Corynebacterium, Brevibacterium, Bacillus, Lactobacillus, Lactococcus, Candida, Pichia, Kluveromyces, Saccharomyces, Bacillus, Escherichia and Clostridium, with further preference for Bacillus flavum, Bacillus lactofermentum, Escherichia coli, Saccharomyces cerevisiae, Kluveromyces lactis, Candida blankii, Candida rugosa, Corynebacterium glutamicum, Corynebacterium efficiens and Pichia postoris, and most preference for Corynebacterium glutamicum. The cells which can be employed in the method of the invention are selected in particular from the group consisting of the wild-type strains Corynebacterium glutamicum ATCC13032, Corynebacterium acetoglutamicum ATCC15806, Corynebacterium acetoacidophilum ATCC13870, Corynebacterium thermoaminogenes FERM BP-1539, Corynebacterium melassecola ATCC17965, Brevibacterium flavum ATCC14067, Brevibacterium lactofermentum ATCC13869 and Brevibacterium divaricatum ATCC14020. Preference is further given to bacteria which are already genetically modified and in which the activity of at least one of the enzymes E₃ to E₂₅ or else one of the enzymatic activities E₁ₐ, E_{1b} or E₂ are increased and, where appropriate, one or more of the enzymatic activities E₂₆ to E₃₈ are diminished. Among these genetically modified cells, particular preference is given to the strain Corynebacterium glutamicum ATCC13032 DM1727 (Georgi et al., Metabolic Engineering 7: 291-301 (2005)) which has an exogenous pyruvate carboxylase with an amino acid mutation at position 458 (proline replaced by serine, see “A novel methodology employing Corynebacterium glutamicum genome information to generate a new L-lysine-producing mutant” (Ohnishi et al., Applied Microbiology and Biotechnology 58: 217-223 (2002)).

Cells able to export beta-alanine from the cell (and thus satisfying condition C)) are preferably those cells which exhibit an efflux of beta-alanine from the cell which is increased by comparison with their wild type, preferably increased by at least 10%, particularly preferably by at least 25%, further preferably by at least 50%, further even more preferably by at least 75%, further preferably by at least 100% and most preferably by at least 500%, maximally preferably by 5000%, particularly preferably by 2500%, this increased efflux preferably being made possible by an increased activity of an enzyme which catalyzes the efflux of beta-alanine out of the cell. Cells able to convert beta-alanine into 3-hydroxy-propionic acid (and thus satisfying condition D)) are in particular those cells in which the activity of at least one, preferably all, of the enzymes E₃ to E₆ described in connection with the cells of the invention, or cells in which the activity of at least one, preferably both, of the enzymes E₇ and E₈ described in connection with the cells of the invention, is increased.

It is further preferred according to the invention for the method for producing genetically modified cells also to include further steps of the method, such as, for instance, increasing the activity of one or more of the enzymes E₉ to E₂₇ described in connection with the cells of the invention, or diminishing the activity of the enzymes E₂₈ to E₄₀ described in connection with the cells of the invention.

A contribution to achieving the objects mentioned at the outset is also provided by the genetically modified cells obtainable by the method described above. These are able, alone or else in combination with other cells, to form 3-hydroxypropionic acid from carbohydrates or from glycerol.

A further contribution to achieving the objects mentioned at the outset is provided by a method for producing 3-hydroxypropionic acid, including the steps of the method
i) contacting the genetically modified cells of the invention which has property c) or C) with a nutrient medium containing carbohydrates or glycerol under conditions under which beta-alanine is formed from the carbohydrates or the glycerol and at least in part reaches the nutrient medium from the cell, so that a beta-alanine-containing nutrient medium is obtained,
ii) contacting the beta-alanine-containing nutrient medium with a further cell which is able to take up the beta-alanine and convert it into 3-hydroxy-propionic acid.

Cells able to take up beta-alanine and convert it into 3-hydroxypropionic acid are particularly preferably those cells in which the activity of at least one, preferably all, of the enzymes E₃ to E₆ or else cells in which the activity of at least one, preferably both, of the enzymes E₇ and E₈ has been increased by comparison with the wild type preferred. Such cells are described for example in WO-A 02/42418 and WO-A 03/62173. Particular preference is further given to cells in which, in addition to these enzymatic activities, there has also been an increase in the activity of enzymes which increase the transport or efflux of beta-alanine into the cells. Particularly preferred in this connection is the GABA transporter GAT-2 and the transport system which is encoded by the cycA gene and is described in Schneider et al., (Appl. Microbiol. Biotechnol. 65: 576-582 (2004)).

The genetically modified cells of the invention can be brought in contact with the nutrient medium, and thus cultured, in step i) of the method continuously or discontinuously in a batch method or in a fed-batch method or repeated fed-batch method for the purpose of producing beta-alanine. A semicontinuous method as described in GB-A 1009370 is also conceivable. A summary of known culturing methods are described in the textbook by Chmiel (“Bioprozesstechnik 1. Einfüzhrung in die Bioverfahrenstechnik” (Gustav Fischer Verlag, Stuttgart, 1991)) or in the textbook by Storhas (“Bioreaktoren und periphere Einrichtungen”, Vieweg Verlag, Brunswick/Wiesbaden, 1994).

The culture medium to be used must satisfy the demands of the respective strains in a suitable manner. Descriptions of culture media for various microorganisms are present in the handbook “Manual of Methods for General Bacteriology” of the American Society for Bacteriology (Washington D.C., USA, 1981).

It is possible to use as carbon source sugars and carbohydrates such as, for example, glucose, sucrose, lactose, fructose, maltose, molasses, starch and cellulose, oils and fats such as, for example, soybean oil, sunflower oil, peanut oil and coconut fat, fatty acids such as, for example, palmitic acid, stearic acid and linoleic acid, alcohols such as, for example, glycerol and ethanol and organic acids such as, for example, acetic acid. These substances can be used singly or as mixture. It is particularly preferred to employ carbohydrates, especially monosaccharides, oligosaccharides or polysaccharides, as described in U.S. Pat. No. 6,01,494 and U.S. Pat. No. 6,136,576, C₅ sugars or glycerol.

It is possible to use as nitrogen source organic nitrogen-containing compounds such as peptone, yeast extract, meat extract, malt extract, corn steep liquor, soybean meal and urea or inorganic compounds such as ammonium sulfate, ammonium chloride, ammonium phosphate, ammonium carbonate and ammonium nitrate. The nitrogen sources can be used singly or as mixture.

It is possible to use as phosphorus source phosphoric acid, potassium dihydrogenphosphate or dipotassium hydrogenphosphate or the corresponding sodium-containing salts. The culture medium must additionally comprise salts of metals such as, for example, magnesium sulfate or iron sulfate, which are necessary for growth. Finally, essential growth promoters such as amino acids and vitamins can be employed in addition to the abovementioned substances. It is moreover possible to add suitable precursors to the culture medium. Said starting materials can be added to the culture in the form of a single batch or be fed in during the culturing in a suitable manner.

The pH of the culture is controlled by employing basic compounds such as sodium (hydrogen)carbonate, sodium hydroxide, potassium hydroxide, ammonia or aqueous ammonia or acidic compounds such as phosphoric acid or sulfuric acid in a suitable manner. Foaming can be controlled by employing antifoams such as, for example, fatty acid polyglycol esters. The stability of plasmids can be maintained by adding to the medium suitable selectively acting substances such as, for example, antibiotics. In order to maintain aerobic conditions, oxygen or oxygen-containing gas mixtures such as, for example, air are introduced into the culture. The temperature of the culture is normally 20° C. to 45° C. and preferably 25° C. to 40° C. Especially on use of cells able to convert glycerol as substrate it may be preferred to employ as cells those cells described in U.S. Pat. No. 6,803,218 and to increase in these cells the activity of the enzymes E₁ₐ or E_{1b} and/or E₂ (and, where appropriate, of the further enzymes E₃ to E₂₀). In this case, the cells can be cultured at temperatures in the range from 40 to 100° C.

At the same time as the formation of beta-alanine, or separate from this step of the method, the beta-alanine-containing nutrient medium is contacted with the further cells which are able to take up beta-alanine and convert it into 3-hydroxypropionic acid. It is possible in this connection for the cells of the invention and the further cells to be cultured together in a nutrient medium so that the beta-alanine released by the cells of the invention is taken up virtually in the nascent state by the further cells and converted into 3-hydroxypropionic acid.

However, it is also conceivable initially to form a beta-alanine-containing nutrient medium, to remove from this the cells of the invention and only then to contact this beta-alanine-containing nutrient medium with the further cells. However, simultaneous culturing of the cells of the invention and the further cells is particularly preferred.

The method of the invention for producing 3-hydroxy-propionic acid may also include as further step iii) of the method the purification of the eventually obtained 3-hydroxypropionic acid from the nutrient medium. This purification can take place by any purification method known to the skilled worker. Thus, for example, sedimentation, filtration or centrifugation methods can be employed in order to remove the cells. The 3-hydroxypropionic acid can be isolated by extraction, distillation, ion exchange, electrodialysis or crystallization from the 3-hydroxypropionic acid-containing nutrient medium which has been freed of cells.

In a particular embodiment of the method of the invention, the 3-hydroxypropionic acid is purified from the nutrient solution continuously, it being further preferred in this connection for the fermentation also to be carried out continuously, so that the overall process from the enzymatic conversion of the precursors to form 3-hydroxypropionic acid up to purification of the 3-hydroxypropionic acid from the nutrient medium can be carried out continuously. For continuous purification of the 3-hydroxypropionic acid from the nutrient medium, the matter is continuously passed through an apparatus for removing the cells employed in the fermentation, preferably through a filter with an exclusion limit in a range from 20 to 200 kDa, in which a solid/liquid separation takes place. It is also conceivable to employ a centrifuge, a suitable sedimentation apparatus or a combination of these apparatuses, it being particularly preferred to remove at least some of the cells initially through sedimentation and subsequently to feed the nutrient medium which has been partly freed of cells to an ultrafiltration or centrifugation apparatus.

The fermentation product which has been enriched in terms of its 3-hydroxypropionic acid content is, after removal of the cells, passed to a preferably multistage separation system. In this separation system there are provided a plurality of successive separation stages from each of which return lines issue and lead back to the second fermentation tank. In addition, discharge lines lead out of the respective separation stages. The individual separation stages can operate on the principle of electrodialysis, reverse osmosis, ultrafiltration or nanofiltration. Normally, there are membrane separation devices in the individual separation stages. The selection of the individual separation stages depends on the nature and extent of the fermentation byproducts and substrate residues.

Besides removal of the 3-hydroxypropionic acid by means of electrodialysis, reverse osmosis, ultrafiltration or nanofiltration, the final product resulting from which is an aqueous 3-hydroxypropionic acid solution, the 3-hydroxypropionic acid can also be removed by extraction methods from the nutrient medium which has been freed of cells, it being possible in this case eventually to obtain pure 3-hydroxypropionic acid. The 3-hydroxypropionic acid can be removed by extraction by adding for example high-boiling organic amines to the nutrient medium in which the 3-hydroxypropionic acid is present as ammonium salt. The mixture obtained in this way is then heated, during which ammonia and water escape and the 3-hydroxypropionic acid is extracted into the organic phase. This method is referred to as salt splitting and is to be found in WO-A 02/090312, the disclosure of which in relation to the removal of 3-hydroxypropionic acid from nutrient media is hereby introduced as reference and forms part of the disclosure of the present application.

A contribution to achieving the objects mentioned at the outset is also provided by a method for producing 3-hydroxypropionic acid including the step of the method of contacting a cell of the invention which has property d) or D) with a nutrient medium containing carbohydrates or glycerol under conditions under which 3-hydroxypropionic acid is formed from the carbohydrates or the glycerol. The culturing takes place in substantially the same way as for the method described above for producing 3-hydroxypropionic acid, although in this case genetically modified cells of the invention able to convert beta-alanine into 3-hydroxypropionic acid are employed. Cells of the invention capable of this have already been described in detail at the outset.

This method for producing 3-hydroxypropionic acid may also include as further step of the method the purification of the 3-hydroxypropionic acid from the nutrient medium.

A further contribution to achieving the objects mentioned at the outset is provided by a method for producing acrylic acid, including the steps of the method
I) production of 3-hydroxypropionic acid by the method described above, where appropriate followed by one or more purification steps,
II) dehydration of the 3-hydroxypropionic acid to form acrylic acid.

The dehydration of the 3-hydroxypropionic acid can in principle be carried out in liquid phase or in the gas phase, with preference for a liquid-phase dehydration. It is further preferred according to the invention for the dehydration to take place in the presence of a catalyst, with the nature of the catalyst employed being dependent on whether a gas-phase or a liquid-phase reaction is carried out. Suitable dehydration catalysts are both acid and alkaline catalysts. Acid catalysts are particularly preferred because of the small tendency to form oligomers. The dehydration catalyst can be employed both as homogeneous and as heterogeneous catalyst. Following the dehydration, an acrylic acid-containing phase is obtained and can be purified where appropriate by further purification steps, in particular by distillation methods, extraction methods or crystallization methods, or else by a combination of these methods.

A further contribution to achieving the objects mentioned at the outset is also provided by a method for producing polyacrylates, including the steps of the method
I) production of 3-hydroxypropionic acid by one of the methods described above, where appropriate followed by one or more purification steps,
II) dehydration of the 3-hydroxypropionic acid to form acrylic acid by the method described above, where appropriate followed by one or more purification steps,
III) free-radical polymerization of the acrylic acid.

The free-radical polymerization of acrylic acid takes place by polymerization methods known to the skilled worker and can be carried out both in an emulsion or suspension and in aqueous solution. It is further possible for further comonomers, especially crosslinkers, to be present during the polymerization. The free-radical polymerization of the acrylic acid obtained in step II) of the method in at least partly neutralized form in the presence of crosslinkers is particularly preferred. This polymerization results in hydrogels which can then be comminuted, ground and, where appropriate, surface-modified, in particular surface-post-crosslinked. The polymers obtained in this way are particularly suitable for use as superabsorbents in hygiene articles such as, for instance, diapers or sanitary napkins.

A contribution to achieving the objects mentioned at the outset is also provided by a method for producing acrylic esters including the steps of the method
I) production of 3-hydroxypropionic acid by one of the methods described above, where appropriate followed by one or more purification steps,
II) dehydration of the 3-hydroxypropionic acid to form acrylic acid by the method described above, where appropriate followed by one or more purification steps,
III) esterification of the acrylic acid.

The esterification of the acrylic acid takes place by esterification methods known to the person skilled in the art, particularly preferably by contacting the acrylic acid obtained in step II) of the method with alcohols, preferably with methanol, ethanol, 1-propanol, 2-propanol, n-butanol, tert-butanol or isobutanol, and heating to a temperature of at least 50° C., particularly preferably at least 100° C. The water formed during the esterification can where appropriate be removed from the reaction mixture by azeotropic distillation through the addition of suitable separation aids.

A further contribution to achieving the objects mentioned at the outset is provided by the use of a cell which is genetically modified in relation to its wild type and which exhibits at least one, preferably both, of properties a) and b):
a) an activity, which is increased by comparison with its wild type, preferably by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of an enzyme E₁ₐ which catalyzes the conversion of pyruvate into oxaloacetate, or of an enzyme E_{1b} which catalyzes the conversion of phosphoenolpyruvate into oxaloacetate, but preferably of an enzyme E₁ₐ which catalyzes the conversion of pyruvate into oxalo-acetate,
b) an activity, which is increased by comparison with its wild type, preferably by at least 10%, particularly preferably by at least 25%, further preferably by at least 50%, further even more preferably by at least 75%, further preferably by at least 100% and most preferably by at least 500%, maximally preferably up to 5000%, particularly preferably up to 2500%, of an enzyme E₂ which catalyzes the conversion of aspartate into β-alanine,
   for producing 3-hydroxypropionic acid.

The present invention is now explained in more detail by means of non-limiting figures and examples.

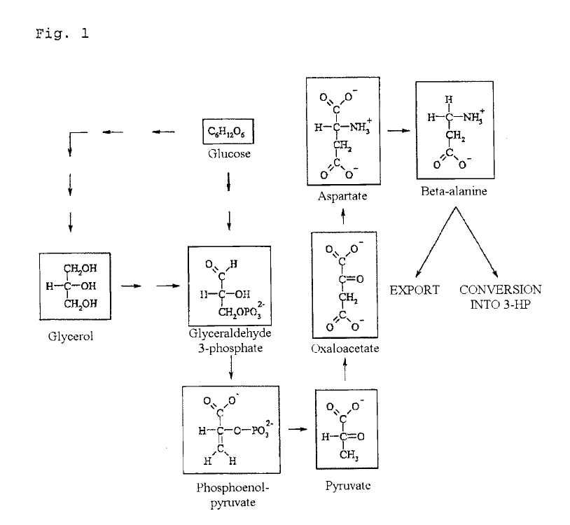

FIG. 1 shows the reaction scheme for forming beta-alanine from glucose or glycerol via pyruvate, oxalo-acetate and aspartate.

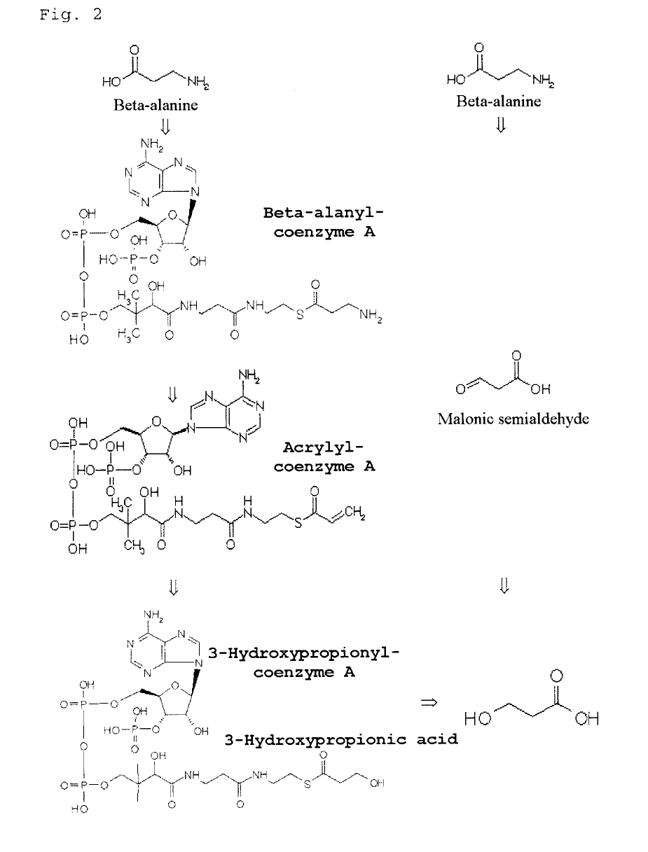

FIG. 2 shows the reaction scheme for forming 3-hydroxypropionic acid from beta-alanine via beta-alanyl-coenzyme A, acrylyl-coenzyme A and 3-hydroxy-propionyl-coenzyme A or via malonic semialdehyde.

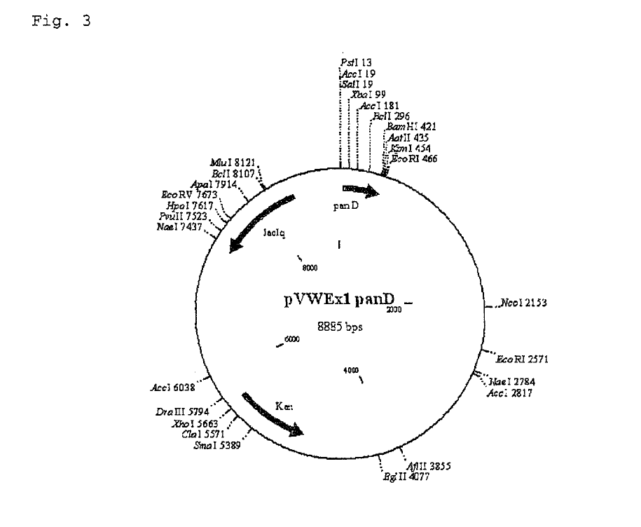

FIG. 3 shows the plasmid vector pVWex1-panD.

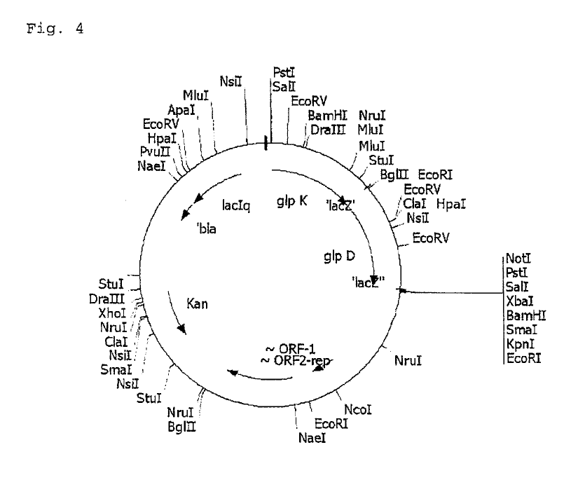

FIG. 4 shows the plasmid vector pVWEx1-glpKD_{E.C}.

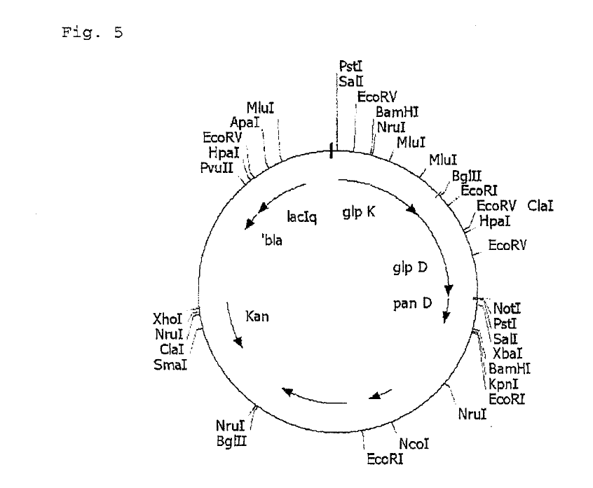

FIG. 5 shows the plasmid vector pVWEx1-panD-glpKD_{E.C}.

### EXAMPLE

A genetically modified cell of the strain Corynebacterium glutamicum in which the heterologous genes glpK, glpD, and the homologous genes pyc and panD were expressed was produced. The procedure for this was as follows:

The starting strains used were the wild-type strain ATCC13032 (deposited at the Deutsche Sammlung von Mikroorganismen und Zellkulturen GmbH, Brunswick, with DSM number 20300) and the strain DM1727. The strain DM1727 was described by Georgi et al. (Metabolic Engineering 7: 291-301 (2005)) and represents a genetically modified Corynebacterium glutamicum strain which exhibits a pyruvate carboxylase activity which is increased in relation to the wild-type strain. This increased activity of the enzyme is attributable to a mutation of the amino acid at position 458 (exchange of proline for serine). Concerning this, see also “A novel methodology employing Corynebacterium glutamicum genome information to generate a new L-lysine-producing mutant” (Ohnishi et al., Applied Microbiology and Biotechnology 58: 217-223 (2002)).

1. Production of the Plasmid Vectors

Firstly, the following two PCR primers were synthesized (underlined nucleotide corresponds to the base pair in the E. coli genom MG1655 (GenBank Reference Sequence NC 000913 (U00096), cleavage sites are emboldened):

| | |
|---|---|
| glpKᵣₑᵥ: | |
| 5′ TCTAGATTATTCGTCGTGTTCTTCCCACGCC | (SEQ. ID NO. 2) |

(the underlined nucleotide corresponds to the base pair 4113737 in the MG1655 genome, and the cleavage site corresponds to an XbaI cleavage site)

glpK_{for}:

(SEQ. ID NO. 3)

5′ GGGACGTCGACAAGGAGATATAGATGACTGAAAAAAAATATATC

(the underlined nucleotide corresponds to the base pair 4115245 in the MG1655 genome, and the cleavage site corresponds to a SalI cleavage site)

The primers corresponded to bases 4113737 to 4113762 and 4115225 to 4115245 of the glpK gene of E. coli. It was possible with these primers by means of PCR by the standard method of Innis et al. (PCR protocols. A guide to methods and applications, 1990, Academic Press) for non-degenerate, homologous primers to amplify a fragment of 1533 base pairs of chromosomal DNA of E. coli which was isolated as described by Eikmanns et al. (Microbiology, 140: 1817-1828 (1994)).

This PCR fragment was cloned into the plasmid vector pGEM-T (Promega Corporation, Madison, Wis., USA) to obtain the plasmid vector pGEM-T-glpK_{E.C.}.

Subsequently, the following two PCR primers were synthesized (underlined nucleotide corresponds to the base pair in the E. coli genome MG1655, cleavage sites are emboldened and the region in italics marks a ribosome binding site):

glpD_{for}:

(SEQ. ID NO. 4)

5′ TCTAGAAAGGAGATATAGATGGAAACCAAAGATCTG

(the underlined nucleotide corresponds to the base pair 3560036 in the MG1655 genome, and the cleavage site corresponds to an XbaI cleavage site)

| | |
|---|---|
| glpDᵣₑᵥ: | |
| 5′ GTTAATTCTAGATTACGACGCCAGCGATAA | (SEQ. ID NO. 5) |

(the underlined nucleotide corresponds to the base pair 3561541 in the MG1655 genome, and the cleavage site corresponds to an XbaI cleavage site)

The primers corresponded to bases 3560036 to 3560053 and 3561524 to 3561541 of the plpD gene of E. coli. With these primers it was possible by means of PCR by means of the standard method of Innis et al. (PCR protocols. A guide to methods and applications, 1990, Academic Press) for non-degenerate, homologous primers to amplify a fragment of 1524 base pairs of chromosomal DNA from E. coli which was isolated as described by Eikmanns et al. (Microbiology 140: 1817-1828 (1994)).

This PCR fragment was cloned into the plasmid vector PGEM-T (Promega Corporation, Madison, Wis., USA) to obtain the plasmid vector pGEM-T-glpD_{E.C.}.

The 1524 base-pair fragment was then cleaved out of the plasmid vector pGEM-T glpD_{E.C.}, with XbalI and cloned into the plasmid vector pGEM-T-glpK_{E.C.} which had been cleaved with SpeI-SAP (SAP=shrimp alkaline phosphatase) to result in the plasmid vector PGEM-T-glpKD_{E.C.}. The glpKD_{E.C.} fragment was then cleaved out with SalI and cloned into the plasmid vector pVWEx1 (this expression plasmid was described by Peters-Wendisch et al., Journal of Molecular Microbiology and Biotechnology 3: 295-300 (2001)) cleaved with SalI to obtain the plasmid vector pVWEx1-glpKD_{E.C.}.

Subsequently, the following two PCR primers were synthesized (underlined nucleotide corresponds to the base pair in the Corynebacterium glutamicum genome (NC003450), cleavage sites are emboldened and regions in italics mark a ribosome binding site):

NCg10133_{for}:

(SEQ. ID NO. 6)

5′ GGACACTAGTAAGGAGATATAGATGCTGCGCACCATCCTC

(the underlined nucleotide corresponds to the base pair 145570 in the genome of Corynebacterium glutamicum, and the cleavage site corresponds to an SpeI cleavage site)

NCg10133ᵣₑᵥ:

(SEQ. ID NO. 7)

5′ CTAAAACGGGTACCCTAAATGCTTCTCGACGTC

(the underlined nucleotide corresponds to the base pair 147980 in the genome of Corynebacterium glutamicum, and the cleavage site corresponds to a KpnI cleavage site)

The primers corresponded to bases 147570 to 147588 and 147964 to 147980 of the panD gene of C. glutamicum. With these primers it was possible by means of PCR by the standard method of Innis et al. (PCR protocols. A guide to methods and applications, 1990, Academic Press) for non-degenerate, homologous primers to amplify a fragment of about 430 base pairs of chromosomal DNA from C. glutamicum which was isolated as described by Eikmanns et al. (Microbiology 140: 1817-1828 (1994)).

This PCR fragment was cloned into the plasmid vector pGEM-T (Promega Corporation, Madison, Wis., USA) to obtain the plasmid vector pGEM-T-panD.

The panD fragment was then cleaved out of pGEM-T-panD using SpeI and KpnI, and cloned into the plasmid vector pVWEx1 which had been cleaved with XbaI and KpnI to obtain the plasmid vector pVWEx1-panD. In addition, the panD fragment cleaved out of pGEM-T-pand by means of speI and KpnI was cloned into the plasmid vector pVWEx1-glpKD_{E.C.} cleaved with XbaI and KpnI to obtain the plasmid vector pVWEx1-panD-glpKD_{E.C.} (SEQ. ID NO. 1).

The plasmid vectors pVWEx1-panD, pVWEx1-glpKD_{E.C.}, and pVWEx1-panD-glpKD_{E.C.} (SEQ. ID NO. 1) are shown in FIGS. 3 to 5.

2. Transformation of Cells

The expression plasmids pVWEx1, pVWEx1-panD_{C.g.}, pVWEx1-glpKD_{E.C.} and pVWEx1-glpKD_{E.C.}panD_{C.g.} were introduced by means of electroporation (according to van der Rest et al., Appl. Microbiol. Biotechnol. 52: 541-545 (1999)) into the starting strains mentioned at the outset.

3. Culturing of the Cells

With glucose as carbon source

The strains transformed with these plasmids were cultured in CGXII medium which was described by Georgi et al. (Metabolic Engineering 7: 291-301 (2005)) and by Marx et al. (U.S. Pat. No. 6,355,454). The medium contained 40 g/kg glucose.

The alpha- or beta-alanine concentration was detected by means of HPLC. The method was described by Georgi et al. (Metabolic Engineering 7: 291-301 (2005)) and by Marx et al. (U.S. Pat. No. 6,355,454). An appropriate standard was employed to identify the alpha-alanine or beta-alanine signal.

After the cells had been cultured for 29 hours, the following concentrations of alpha-alanine and beta-alanine were measured in the nutrient medium:

| C. glutamicum strain | alpha-alanine | beta-alanine |
|---|---|---|
| ATCC13032 (pVWEx1) | 20.4 mM | <1 mM |
| ATCC13032 (pVWEx1-panD_{C.g.}) | 15.5 mM | 23.9 mM |
| DM1727 (pVWEx1-panD_{C.g.}) | 5.0 mM | 18.6 mM |

3.2. With Glycerol as Carbon Source

The strains transformed with these plasmids were cultured in CGXII medium which was described by Georgi et al. (Metabolic Engineering 7: 291-301 (2005)) and by Marx et al. (U.S. Pat. No. 6,355,454). The medium contained 9 g/kg glycerol.

The alpha- or beta-alanine concentration was detected by means of HPLC. The method was described by Georgi et al. (Metabolic Engineering 7: 291-301 (2005)) and by Marx et al. (U.S. Pat. No. 6,355,454). An appropriate standard was employed to identify the alpha-alanine or beta-alanine signal.

After the cells had been cultured for 24 hours, the following concentrations of alpha-alanine and beta-alanine were measured in the nutrient medium:

| C. glutamicum strain | alpha-alanine | beta-alanine |
|---|---|---|
| ATCC13032 (pVWEx1-glpKD_{E.c.}) | 2.8 mM | <1 mM |
| ATCC13032 (pVWEx1-glpKD_{E.c.}panD_{C.g.}) | 3 mM | 0.5 mM |
| DM1727 (pVWEx1-glpKD_{E.c.}panD_{C.g.}) | 0.4 mM | 0.5 mM |

1. A cell which is genetically modified in relation to its wild type and which exhibits at least one of the properties a) or b):
a) an increased activity by comparison with its wild type of an enzyme E₁ₐ which catalyzes the conversion of pyruvate into oxaloacetate, or of an enzyme E_{1b} which catalyzes the conversion of phosphoenolpyruvate into oxaloacetate,
b) an increased activity by comparison with its wild type of an enzyme E₂ which catalyzes the conversion of aspartate into beta-alanine,
wherein, besides properties a) and b), the cell displays at least one of the properties c) or d)
c) the genetically modified cell has the ability to release beta-alanine from the cell,
d) the genetically modified cell has the ability to convert beta-alanine into 3-hydroxypropionic acid.

2. The cell as claimed in claim 1, wherein the enzyme E₁ is a pyruvate carboxylase.

3. The cell as claimed in claim 1, wherein the increased activity of the enzyme E₁ results from a mutation of the pyruvate carboxylase gene of the wild type of the cell.

4. The cell as claimed in claim 1, wherein the enzyme E₂ is an aspartate decarboxylase.

5. The cell as claimed in claim 1 which exhibits property d), wherein the cell exhibits an increased activity by comparison with its wild type of at least one of the following enzymes E₃ to E₆:
of an enzyme E₃ which catalyzes the conversion of beta-alanine into beta-alanyl-coenzyme A,
of an enzyme E₄ which catalyzes the conversion of beta-alanyl-coenzyme A into acrylyl-coenzyme-A,
of an enzyme E₅ which catalyzes the conversion of acrylyl-coenzyme A into 3-hydroxypropionyl-coenzyme A,
of an enzyme E₆ which catalyzes the conversion of 3-hydroxypropionyl-coenzyme A into 3-hydroxypropionic acid.

6. The cell as claimed in claim 5, wherein the enzyme
E₃ is a coenzyme A transferase or coenzyme A synthetase,
E₄ is a beta-alanyl-coenzyme A ammonium-lyase,
E₅ is a 3-hydroxypropionyl-coenzyme A dehydratase, and
E₆ is a coenzyme A transferase, 3-hydroxypropionyl-coenzyme A hydrolase or 3-hydroxybutyryl-coenzyme A hydrolase.

7. The cell as claimed in claim 1, which exhibits property d), wherein the cell exhibits an increased activity by comparison with its wild type of at least one of the following enzymes E₇ and E₈:
of an enzyme E₇ which catalyzes the conversion of beta-alanine into malonic semialdehyde,
of an enzyme E₈ which catalyzes the conversion of malonic semialdehyde into 3-hydroxypropionic acid.

8. The cell as claimed in claim 7, wherein the enzyme
E₇ is a beta-alanine-2-oxoglutarate aminotransferase and
E₈ is a 3-hydroxypropionyl dehydrogenase or 3-hydroxybutyrate dehydrogenase.

9. The cell as claimed in claim 1, wherein the cell exhibits a phosphoglucoisomerase activity which is reduced by comparison with its wild type.

10. A method for producing a genetically modified cell which displays at least one of properties c) and d):
c) the genetically modified cell has the ability to release beta-alanine from the cell,
d) the genetically modified cell has the ability to convert beta-alanine into 3-hydroxypropionic acid.
comprising at least one, of steps A) and B):
A) increasing the activity of an enzyme E₁ₐ which catalyzes the conversion of pyruvate into oxaloacetate, or of an enzyme E_{1b} which catalyzes the conversion of phosphoenolpyruvate into oxaloacetate, in a cell, or
B) increasing the activity of an enzyme E₂ which catalyzes the conversion of aspartate into beta-alanine in a cell.

11. The method as claimed in claim 10, wherein the enzyme E₁ₐ is a pyruvate carboxylase and the enzyme E_{1b} is a phosphoenolpyruvate carboxylase.

12. The method as claimed in claim 10, wherein the enzyme E₂ is an aspartate decarboxylase.

13. A cell obtainable by a method as claimed in claim 10.

14. A method for producing 3-hydroxypropionic acid, comprising:
i) contacting a first cell as claimed in claim 1 which exhibits property c) with a nutrient medium containing carbohydrates or glycerol under conditions under which beta-alanine is formed from the carbohydrates or the glycerol and at least in part reaches the nutrient medium from the cell, so that a beta-alanine-containing nutrient medium is obtained, and
ii) contacting the beta-alanine-containing nutrient medium with a second cell which has the ability to take up the beta-alanine and convert it into 3-hydroxypropionic acid.

15. A method for producing 3-hydroxypropionic acid comprising contacting a cell as claimed in claim 1 which exhibits property d) with a nutrient medium containing carbohydrates or glycerol under conditions under which 3-hydroxypropionic acid is formed from the carbohydrates or the glycerol.

16. The method for as claimed in claim 14, further comprising:
iii) dehydrating said 3-hydroxypropionic acid to form acrylic acid.

17. The method as claimed in claim 14 further comprising:
iii) dehydrating said 3-hydroxypropionic acid to form acrylic acid, and
iv) polymerizing said acrylic acid via radical polymerization.

18. The method as claimed in claim 15 further comprising:
iii) dehydrating said 3-hydroxypropionic acid to form acrylic acid, and
v) esterifying the acrylic acid.

19. A method of producing 3-hydroxypropionic acid, comprising: culturing a genetically modified cell in relation to its wild type and which exhibits at least one of properties a) or b)
a) an increased activity by comparison with its wild type of an enzyme E₁ₐ which catalyzes the conversion of pyruvate into oxaloacetate, or of an enzyme E_{1b} which catalyzes the conversion of phosphoenolpyruvate into oxaloacetate,
b) an increased activity by comparison with its wild type of an enzyme E₂ which catalyzes the conversion of aspartate into beta-alanine,
to produce said 3-hydroxypropionic acid.


SEQUENCE LISTING (7 sequences)

SEQ ID NO: 1 - DNA, 11959 bp, Artificial Sequence
  Description of Artificial Sequence Synthetic plasmid pVWEx1-glpKD-panD sequence
aagcttgcatgcctgcaggtcgacaaggagatatagatgactgaaaaaaaatatatcgttgcgctcgaccagggcaccaccagctcccgcgcggtcgtaatggatcacgatgccaatatcattagcgtgtcgcagcgcgaatttgagcaaatctacccaaaaccaggttgggtagaacacgacccaatggaaatctgggccacccaaagctccacgctggtagaagtgctggcgaaagccgatatcagttccgatcaaattgcagctatcggtattacgaaccagcgtgaaaccactattgtctgggaaaaagaaaccggcaagcctatctataacgccattgtctggcagtgccgtcgtaccgcagaaatctgcgagcatttaaaacgtgacggtttagaagattatatccgcagcaataccggtctggtgattgacccgtacttttctggcaccaaagtgaagtggatcctcgaccatgtggaaggctctcgcgagcgtgcacgtcgtggtgaattgctgtttggtacggttgatacgtggcttatctggaaaatgactcagggccgtgtccatgtgaccgattacaccaacgcctctcgtaccatgttgttcaacatccataccctggactgggacgacaaaatgctggaagtgctggatattccgcgcgagatgctgccagaagtgcgtcgttcttccgaagtatacggtcagactaacattggcggcaaaggcggcacgcgtattccaatctccgggatcgccggtgaccagcaggccgcgctgtttggtcagttgtgcgtgaaagaagggatggcgaagaacacctatggcactggctgctttatgctgatgaacactggcgagaaagcggtgaaatcagaaaacggcctgctgaccaccatcgcctgcggcccgactggcgaagtgaactatgcgttggaaggtgcggtgtttatggcaggcgcatccattcagtggctgcgcgatgaaatgaagttgattaacgacgcctacgattccgaatatttcgccaccaaagtgcaaaacaccaatggtgtgtatgtggttccggcatttaccgggctgggtgcgccgtactgggacccgtatgcgcgcggggcgattttcggtctgactcgtggggtgaacgctaaccacattatacgcgcgacgctggagtctattgcttatcagacgcgtgacgtgctggaagcgatgcaggccgactctggtatccgtctgcacgccctgcgcgtggatggtggcgcagtagcaaacaatttcctgatgcagttccagtccgatattctcggcacccgcgttgagcgcccggaagtgcgcgaagtcaccgcattgggtgcggcctatctcgcaggcctggcggttggcttctggcagaacctcgacgagctgcaagagaaagcggtgattgagcgcgagttccgtccaggcatcgaaaccactgagcgtaattaccgttacgcaggctggaaaaaagcggttaaacgcgcgatggcgtgggaagaacacgacgaataaatcactagaaaggagatatagatggaaaccaaagatctgattgtgatagggggcggcatcaatggtgctggtatcgcggcagacgccgctggacgcggtttatccgtgctgatgctggaggcgcaggatctcgcttgcgcgacctcttccgccagttcaaaactcattcacggtggcctgcgctaccttgagcactatgaattccgcctggtcagcgaggcgctggctgaacgtgaagtgctgctgaaaatggccccgcatatcgccttcccgatgcgttttcgcctgccacatcgtccgcatctgcgcccggcgtggatgattcgcattggtctgtttatgtacgatcatctgggtaaacgcaccagcttgccgggatcaactggtttgcgttttggcgcaaattcagtgttaaaaccggaaattaagcgcggattcgaatattctgactgttgggtagacgacgcccgtctggtactcgccaacgcccagatggtggtgcgtaaaggcggcgaagtgcttactcggactcgcgccacctctgctcgccgcgaaaacggcctgtggattgtggaagcggaagatatcgataccggcaaaaaatatagctggcaagcgcgcggcttggttaacgccaccggcccgtgggtgaaacagttcttcgacgacgggatgcatctgccttcgccttatggcattcgcctgatcaaaggcagccatattgtggtgccgcgcgtgcatacccagaagcaagcctacattctgcaaaacgaagataaacgtattgtgttcgtgatcccgtggatggacgagttttccatcatcggcactaccgatgtcgagtacaaaggcgatccgaaagcggtgaagattgaagagagtgaaatcaattacctgctgaatgtgtataacacgcactttaaaaagcagttaagccgtgacgatatcgtctggacctactccggtgtgcgtccgctgtgtgatgatgagtccgactcgccgcaggctattacccgtgattacacccttgatattcatgatgaaaatggcaaagcaccgctgctgtcggtattcggcggtaagctgaccacctaccgaaaactggcggaacatgcgctggaaaaactaacgccgtattatcagggtattggcccggcatggacgaaagagagtgtgctaccgggtggcgccattgaaggcgaccgcgacgattatgccgctcgcctgcgccgccgctatccgttcctgactgaatcgctggcgcgtcattacgctcgcacttacggcagcaacagcgagctgctgctcggcaatgcgggaacggtaagcgatctcggggaagatttcggtcatgagttctacgaagcggagctgaaatacctggtggatcacgaatgggtccgccgcgccgacgacgccctgtggcgtcgcacaaaacaaggcatgtggctaaatgcggatcaacaatctcgtgtgagtcagtggctggtggagtatacgcagcagaggttatcgctggcgtcgtaatctagtgcggccgcctgcaggtcgactctagtaaggagatatagatgctgcgcaccatcctcggaagtaagattcaccgagccactgtcactcaagctgatctagattatgttggctctgtaaccatcgacgccgacctggttcacgccgccggattgatcgaaggcgaaaaagttgccatcgtagacatcaccaacggcgctcgtctggaaacttatgtcattgtgggcgacgccggaacgggcaatatttgcatcaatggtgccgctgcacaccttattaatcctggcgatcttgtgatcatcatgagctaccttcaggcaactgatgcggaagccaaggcgtatgagccaaagattgtgcacgtggacgccgacaaccgcatcgttgcgctcggcaacgatcttgcggaagcactacctggatccgggcttttgacgtcgagaagcatttagggtaccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgatccccctgcggcgtcgctgatcgccctcgcgacgttgtgcgggtggcttgtccctgagggcgctgcgacagatagctaaaaatctgcgtcaggatcgccgtagagcgcgcgtcgcgtcgattggaggcttcccctttggttgacggtcttcaatcgctctacggcgatcctgacgcttttttgttgcgtaccgtcgatcgttttatttctgtcgatcccgaaaaagtttttgccttttgtaaaaaacttctcggtcgccccgcaaattttcgattccagattttttaaaaaccaagccagaaatacgacacaccgtttgcagataatctgtctttcggaaaaatcaagtgcgatacaaaatttttagcacccctgagctgcgcaaagtcccgcttcgtgaaaattttcgtgccgcgtgattttccgccaaaaactttaacgaacgttcgttataatggtgtcatgaccttcacgacgaagtaccaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggcagcgccaggaggacgcacagtagtggaggatcgaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaaagatcttcaaagccactgccgcgactccgcttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttgctcgcggcggtgccgctggttgcgcttggcttgaccgacttgatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgccgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaaaagatatgaccatgattacgcc

SEQ ID NO: 2 - DNA, 31 bp, Artificial Sequence
  Description of Artificial Sequence Synthetic primer
tctagattattcgtcgtgttcttcccacgcc

SEQ ID NO: 3 - DNA, 44 bp, Artificial Sequence
  Description of Artificial Sequence Synthetic primer
gggacgtcgacaaggagatatagatgactgaaaaaaaatatatc

SEQ ID NO: 4 - DNA, 36 bp, Artificial Sequence
  Description of Artificial Sequence Synthetic primer
tctagaaaggagatatagatggaaaccaaagatctg

SEQ ID NO: 5 - DNA, 30 bp, Artificial Sequence
  Description of Artificial Sequence Synthetic primer
gttaattctagattacgacgccagcgataa

SEQ ID NO: 6 - DNA, 40 bp, Artificial Sequence
  Description of Artificial Sequence Synthetic primer
ggacactagtaaggagatatagatgctgcgcaccatcctc

SEQ ID NO: 7 - DNA, 33 bp, Artificial Sequence
  Description of Artificial Sequence Synthetic primer
ctaaaacgggtaccctaaatgcttctcgacgtc